(19) United States
(12) Patent Application Publication
(10) Pub. No.: US 2009/0305236 A1
(43) Pub. Date: Dec. 10, 2009

(54) METHODS OF ENRICHING FETAL CELLS

(75) Inventors: Ralph Michael Boehmer, St. Kilda West (AU); Richard Allman, Hampton (AU)

Correspondence Address: WILMERHALE/BOSTON, 60 STATE STREET, BOSTON, MA, 02109, US

(73) Assignee: GENETIC TECHNOLOGIES LIMITED, Fitzroy, VC (AU)

(21) Appl. No.: 11/914,107

(22) PCT Filed: May 11, 2006
(86) PCT No.: PCT/AU06/00617

§ 371 Date: Sep. 24, 2008

Related U.S. Application Data
(60) Provisional application No. 60/679,745, filed on May 11, 2005.
(60) Provisional application No. 60/689,745, filed on Jun. 9, 2005.
(60) Provisional application No. 60/725,365, filed on Oct. 11, 2005.

(51) Int. Cl. C12Q 1/68; C12N 5/00; C12Q 1/48

(52) U.S. Cl. 435/6; 435/325; 435/15

(57) ABSTRACT

The present invention relates to methods of enriching fetal cells from a pregnant female. The present invention relates to removing, from a sample, cells that comprise at least one MHC molecule. The present invention also relates to methods that rely on using telomerase, mRNA encoding components thereof, as well as telomere length, as markers for fetal cells. Enriched fetal cells can be used in a variety of procedures including, detection of a trait of interest such as a disease trait, or a genetic predisposition thereto, gender typing and parentage testing.

### FIELD OF THE INVENTION

The present invention relates to methods of enriching fetal cells from a pregnant female. Enriched fetal cells can be used in a variety of procedures including, detection of a trait of interest such as a disease trait, or a genetic predisposition thereto, gender typing and parentage testing.

### BACKGROUND OF THE INVENTION

Fetal testing for chromosomal abnormalities is often performed on cells obtained using amniocentesis, or alternatively, Chorionic Villus Sampling (CVS). Amniocentesis is a procedure used to retrieve fetal cells from the fluid that surrounds the fetus. This relatively invasive procedure is performed after the 12th week of pregnancy. There is about 0.5% increased risk of miscarriage following amniocentesis. CVS is a prenatal test in which cells surrounding an embryo are removed in order to examine the chromosomes. CVS is relatively less invasive, and can be performed as early as 10 weeks from conception. There is about 1% increased risk of miscarriage following CVS.

Fetal therapy is in its very early stages and the possibility of very early tests for a wide range of disorders would undoubtedly greatly increase the pace of research in this area. Current fetal surgical techniques have improved, making fetal surgery for some genetic problems like spina bifida and cleft palate very feasible. In addition, relatively simple effective fetal treatment is currently available for other disorders such as 21-hydroxylase deficiency (treatment with dexamethasone) and holocarboxylase synthetase (treatment with biotin) deficiencies, as long as detection can take place early enough.

At least some fetal cell types such as platelets, trophoplasts, erythrocytes and leucocytes have been shown to cross the placenta and circulate in maternal blood (Douglas et al., 1959; Schroder, 1975). Maternal blood represents a non-invasive source of fetal cell types, however the isolation of fetal cells from maternal blood is hampered by the scarcity of such fetal cells in the maternal circulation, as well as the lack of a marker that identifies all fetal cells, rather than merely a sub-population. A variety of methods have been proposed for isolation or enrichment of fetal cells in maternal blood. These methods include centrifugation techniques, immunoaffinity techniques, and fluorescent in situ hybridization (FISH) methods. However, these methods suffer from a number of deficiencies.

A fetal specific antibody is yet to be identified which can be used to reliably and reproducibly enrich fetal cells. This problem can be overcome with the method described by Simons (U.S. Pat. No. 5,153,117 and U.S. Pat. No. 5,447,842), based on a negative selection approach that does not require knowledge about fetal cell types and fetal cell numbers. However, the Simons method is operationally difficult and expensive to perform, due to the need to HLA type the mother, as well as due to the fact that high-quality specific HLA antibodies are not commercially available.

There is a need in the art for new methods for the enrichment and identification of fetal cells.

### SUMMARY OF THE INVENTION

It is generally considered that Class I Major Histocompatibility Complex (MHC) molecules (human Class I MHC molecules are also known in the art as Class I Human Leukocyte Antigens (HLA)) are expressed on most, if not all, nucleated cell types. Notably, at least the Class I MHC molecules HLA-G and HLA-C have been found to be expressed on some types of fetal trophoblasts (Shorter et al., 1993; King et al., 1996). However, it has surprisingly been found that depleting a sample using an agent which binds MHC molecules results in an enriched population of fetal cells. Furthermore, it has been determined that telomerase and telomeres can be considered as a marker of fetal cells. This enables these molecules to be targeted in procedures for detecting and isolating fetal cells. When combined together, these procedures enhance the purity of enriched fetal cell populations.

Accordingly, in a first aspect the present invention provides a method of enriching fetal cells from a sample, the method comprising

i) depleting maternal cells by removing cells that express at least one MHC molecule on their surface, and

ii) selecting fetal cells by
a) selecting cells that express telomerase, and/or
b) selecting cells based on telomere length.

Steps i) and ii) can be performed in any order. Thus, one step may be performed on the sample obtained from the mother, and the other step on the remaining cell population. Alternatively, the steps may be performed simultaneously.

In another aspect, the present invention provides a method of enriching fetal cells from a sample, the method comprising removing from the sample cells that express at least one MHC molecule on their surface.

Preferably, the MHC molecule is a Class I MHC molecule.

In a further preferred embodiment, all cells expressing at least one Class I MHC molecule are removed.

In a particularly preferred embodiment, the Class I MHC molecule is HLA-A. In another preferred embodiment, the Class I MHC molecule is HLA-B. In a further preferred embodiment, the Class I MHC molecule is HLA-A and HLA-B.

An advantage of the above aspects of the invention when compared to that of Simons (U.S. Pat. No. 5,153,117) is that it is not necessary to determine the genotype of MHC alleles of the mother, father and/or fetus. Thus, in a particularly preferred embodiment, the genotype of an MHC allele is not determined for the mother, father and/or fetus. More preferably, the genotype of an MHC allele is not determined for the mother.

In another embodiment, the method comprises

i) contacting cells in the sample with an agent that binds at least one MHC molecule, and

ii) removing cells bound by the agent.

In a further preferred embodiment, the method comprises contacting the sample with i) an agent that binds at least one Class I MHC molecule, and ii) an agent that binds at least one Class II MHC molecule.

In another preferred embodiment, the agent binds:

i) a monomorphic determinant of HLA-A molecules,

ii) a monomorphic determinant of HLA-B molecules, or

iii) a monomorphic determinant of HLA-A and HLA-B molecules.

In one embodiment, the agent does not bind HLA-C.

In another embodiment, the agent binds a monomorphic determinant of HLA-A, HLA-B and HLA-C molecules. Preferably, the agent that binds a monomorphic determinant of HLA-A, HLA-B and HLA-C molecules is used at sub-saturating concentrations.

In a further embodiment, more than two agents are used which bind different isotypes of the same class or sub-class of MHC molecule. Preferably, collectively the agents bind all isotypes (alleles) of the same class or sub-class of MHC molecule.

In one embodiment, the two agents are an antibody that binds HLA-Bw4 and an antibody that binds HLA-Bw6.

Compounds have been shown to associate in situ with MHC molecules, and hence these compounds can be targeted using the methods of the invention. Accordingly, in another embodiment, the method comprises

i) contacting cells in the sample with an agent that binds a compound that associates with an MHC molecule, and

ii) removing cells bound by the agent.

For example, the compound could be a ligand, for example a protein ligand, that binds an MHC molecule.

The binding of the agent to a maternal cell can be detected directly or indirectly. Direct detection relies on the agent being bound to a detectable label or isolatable label. Indirect detection relies on a further factor, for example a detectably labelled secondary antibody, which binds the agent/maternal cell complex. Preferably, the label is selected from, but not limited to, the group consisting of: a fluorescent label, a radioactive label, a paramagnetic particle (such as a magnetic bead), a chemiluminescent label, a label that is detectable by virtue of a secondary enzymatic reaction, and a label that is detectable by virtue of binding to a molecule.

Labelled cells can be removed from the sample using any technique known in the art. In one embodiment, the step of removing cells comprises detecting the label and removing the labeled cells.

In a further embodiment, the detectable label or isolatable label is a fluorescent label, wherein the step of removing cells comprises performing fluorescence activated cell sorting.

In another embodiment, the detectable label or isolatable label is a paramagnetic particle such as a magnetic bead, wherein the step of removing cells comprises exposing the labelled cells to a magnetic field.

The agent can be any compound which specifically binds MHC expressed on the surface of a maternal cell. Typically, the agent will be an antibody or antibody fragment.

In another embodiment, the maternal cells bound by an antibody which binds an MHC molecule are removed by killing the cells using complement-dependent lysis.

In another aspect, the present invention provides a method of enriching fetal cells from a sample, the method comprising selecting cells from the sample that express telomerase.

Telomerase is a protein/RNA complex. In one embodiment, the method comprises detecting a protein component of telomerase. Preferably, the protein component is telomere reverse transcriptase (TERT). Examples of other proteins which may form part of the telomerase protein/RNA complex are: TEP-1 (telomerase associated protein-1) and 14-3-3 protein.

A protein component of telomerase can be detected using any technique known in the art. Preferably, the cell is exposed to a polypeptide (more preferably, an antibody) which binds telomerases, especially TERT. Using an antibody as an example, the antibody bound to telomerase may be detected directly or indirectly. Direct detection relies on the antibody being detectably labelled. Indirect detection relies on a further factor, for example a detectably labelled secondary antibody, which binds the anti-telomerase antibody/telomerase complex.

In another embodiment, the method comprises detecting an RNA component of telomerase. In yet another embodiment, the method comprises detecting an mRNA encoding a protein component of telomerase.

RNA/mRNA can be detected using any technique known in the art. Typically, the cells are exposed to a labelled probe which hybridizes to the RNA/mRNA. The probe can be of any length or structure as long as it is capable of hybridizing the target RNA or mRNA.

Telomeres prior to birth can be considered to be at maximum length. After birth, with each cell division, they get progressively shorter. Telomeres generally remain until death, however, they just get shorter with time. It has been determined that telomeres are attractive targets to use in identifying fetal cells, (1) because they provide an age-discriminant for cell selection, namely young cells can be separated from older cells (fetal from maternal), and (2) because probes can be designed with a relatively low coefficient of variation and good signal:noise ratio.

Thus, in yet another aspect, the present invention provides a method of enriching fetal cells from a sample, the method comprising selecting cells from the sample based on telomere length.

In one embodiment, the method comprises contacting cells with a detectably labelled probe that binds telomeres.

In another embodiment, about 1 to about 100 cells, more preferably about 1 to about 20 cells and even more preferably about 1 to about 10 cells, are selected, wherein the selected cells have been bound by more probe than the other cells in the sample. In this embodiment, a probe is used that will bind in approximate proportion (by number) to the length of the telomere. Thus, the selected cells are the most intensely labelled cells.

The sample can be obtained from any source known in the art to potentially contain fetal cells. Examples include, but are not limited to, blood, cervical mucous or urine. Preferably, the sample is maternal blood.

When the sample is maternal blood it is preferred that the method further comprises isolating from the maternal blood sample a cell fraction comprising nucleated cells.

In some cases, particularly when performing procedures which detect RNA or DNA, it is preferred that the cells are fixed and permeabilized.

Fetal cell enrichment using the methods of the invention may be further enhanced by negatively selecting for cells that express at least one other maternal cell marker. As outlined above, this marker may be an MHC molecule. In an embodiment, the method further comprises removing from the sample red blood cells, lymphocytes, and/or cancer cells. In a particularly preferred embodiment, the method further comprises removing hemopoietic cells from the sample. Preferably, the method further comprises contacting cells in the sample with an agent that binds a hemopoietic cell.

Examples of hemopoietic cells that can be removed include, but are not limited to, T cells, a B cells, macrophages, neutrophils, dendritic cells and/or basophils.

Preferably, the agent binds a cell surface protein of the cell. Such cell surface proteins are known to those skilled in the art. Examples of cell surface proteins include, but are not limited to, CD3, CD4, CD8, CD10, CD14, CD15, CD45 and CD56.

In a particularly preferred embodiment, the method further comprises contacting cells in the sample with an agent that binds CD45, and removing cells bound by the agent that binds CD45. Such embodiments can be performed using similar techniques to those described herein for depletion using an agent which binds at least one MHC molecule.

The methods of the invention can also be used in combination with further methods of positively selecting for fetal cells by targeting molecules expressed by fetal cells but not by (or only a small proportion of) maternal cells. Thus, in a further embodiment, the method further comprises contacting the cells with an agent that binds fetal cells, and selecting cells bound by the agent that binds fetal cells. Examples of such markers include, but are not limited to, trophoblast specific proteins, fetal or embryonal hemoglobin, and fetal nucleated red blood cell specific proteins.

The sample can be obtained during any stage of pregnancy. If the sample is to be screened to determine if the fetus has a genetic defect, the detection of which may lead to the pregnancy being terminated, it is preferred that the sample is obtained from the mother in the first trimester of pregnancy, preferably between week 8 and week 12.

The labelled fetal cells can be selected using any method known in the art. In many instances the procedure for selection is linked to the nature of the label. For example, where the label used emits a fluorescent signal the cells can be selected by, but not limited to, fluorescence activated cell sorting, fluorescence microscopy, or laser microdissection.

In another aspect, the present invention provides a method of detecting a fetal cell(s) in a sample, the method comprising analysing a candidate cell for the expression of telomerase.

In a further aspect, the present invention provides a method of detecting a fetal cell(s) in a sample, the method comprising analysing a candidate cell for the presence of telomeres and/or analysing the length of the telomeres in a candidate cell.

In a further aspect, the present invention provides an enriched population of fetal cells obtained by a method according to the invention.

In another aspect, the present invention provides a composition comprising fetal cells of the invention, and a carrier.

In yet another aspect, the present invention provides for the use of an agent that binds at least one MHC molecule, and/or an agent that binds a compound that associates with an MHC molecule, for enriching fetal cells from a sample.

In another aspect, the present invention provides for the use of an agent that binds telomerase for enriching fetal cells from a sample.

In yet a further aspect, the present invention provides for the use of an agent that binds telomeres for enriching fetal cells from a sample.

Fetal cells enriched/detected using a method of the invention can be used to analyse the genotype of the fetus. Thus, in another aspect, the present invention provides a method for analysing the genotype of a fetal cell at a locus of interest, the method comprising

i) obtaining enriched fetal cells using a method according to the invention and/or detecting a fetal cell using a method of the invention, and

ii) analysing the genotype of at least one fetal cell at a locus of interest.

The genotype of the fetus can be determined using any technique known in the art. Examples include, but are not limited to, karyotyping, hybridization based procedures, and/or amplification based procedures.

The genotype of a fetal cell can be analysed for any purpose. Typically, the genotype will be analysed to detect the likelihood that the offspring will possess a trait of interest. Preferably, the fetal cell is analysed for a genetic abnormality linked to a disease state, or predisposition thereto. In one embodiment, the genetic abnormality is in the structure and/or number or chromosomes. In another embodiment, the genetic abnormality encodes an abnormal protein. In another embodiment, the genetic abnormality results in decreased or increased expression levels of a gene.

In at least some instances, the enrichment methods of the invention will not result in a pure fetal cell population. In other words, some maternal cells may remain. Thus, in a preferred embodiment the methods of diagnosis (determination, analysis etc) further comprises identifying a cell as a fetal cell. This analysis may positively identify maternal or fetal cells. In the case of positively identifying maternal cells, the non-labelled cells will be fetal cells. Alternatively, both maternal and fetal cells are positively identified using different selectable markers, or a marker that results in a different level of signal between maternal and fetal cells is used. These procedures can be performed using any technique known in the art. For example, for male fetal cells a Y-chromosome specific probe can be used. In another example, telomere length is analysed. In a further embodiment, maternal cells are identified using an agent, such as an antibody, that binds a Class I MHC molecule. Other methods suitable to perform this embodiment are described herein.

The enriched/detected fetal cells can be used to determine the sex of the fetus. As a result, in a further aspect the present invention provides a method of determining the sex of a fetus, the method comprising

i) obtaining enriched fetal cells using a method according to the invention and/or detecting a fetal cell using a method of the invention, and

ii) analysing of at least one fetal cell to determine the sex of the fetus.

The analysis of the fetal cells to determine the sex of the fetus can be performed using any technique known in the art. For example, Y-chromosome specific probes can be used, and/or the cells karyotyped.

The enriched fetal cells can also be used to identify the father of the fetus. Accordingly, in a further aspect, the present invention provides a method of determining the father of a fetus, the method comprising

i) obtaining enriched fetal cells using a method according to the invention and/or detecting a fetal cell using a method of the invention,

ii) determining the genotype of the candidate father at one or more loci,

iii) determining the genotype of the fetus at one or more of said loci, and

iv) comparing the genotypes of ii) and iii) to determine the probability that the candidate father is the biological father of the fetus.

Whilst in some cases it may not be essential that the genotype of the mother also be analysed, for accuracy it is preferred that the method further comprises determining the genotype of the mother at one or more of said loci.

Analysis of the genotype of the candidate father, fetus or mother can be performed using any technique known in the art. One preferred technique is performing DNA fingerprinting analysis using probes/primers which hybridize to tandemly repeated regions of the genome. Another technique is to analyse the HLA/MHC region of the genome.

In a further aspect, the present invention provides a kit for enriching fetal cells from a sample, the kit comprising

i) an agent that binds at least one MHC molecule, and/or an agent that binds a compound that associates with an MHC molecule, and/or an agent that binds a hemopoietic cell, and

ii) a molecule which binds to telomerase, and/or which hybridizes to a polynucleotide encoding a protein component of said telomerase, and/or which hybridizes to telomeres.

In yet another aspect, the present invention provides a kit for enriching fetal cells from a sample, the kit comprising an agent that binds at least one MHC molecule, and/or an agent that binds a compound that associates with an MHC molecule, and/or an agent that binds a hemopoietic cell.

Preferably, the agent that binds at least one MHC molecule is an antibody.

In another embodiment, the kit comprises

i) an agent that binds all HLA-A molecules,

ii) an agent that binds all HLA-B molecules, and/or

iii) an agent that binds all HLA-A and HLA-B molecules.

Preferably, at least one agent is linked to a magnetic bead.

In yet another aspect, the present invention provides a kit for detecting a fetal cell, the kit comprising a molecule which binds to telomerase, and/or which hybridizes to a polynucleotide encoding a protein component of said telomerase, and/or which hybridizes to telomeres.

Preferably, the molecule is selected from the group consisting of; an anti-telomerase antibody, a polynucleotide which hybridizes to mRNA encoding a protein component of telomerase, a polynucleotide which hybridizes to an RNA component of telomerase, or a polynucleotide which hybridizes to telomeric DNA on the chromosome.

Preferably, the molecule is detectably labelled.

In a further aspect, the present invention provides a kit for detecting a genetic abnormality in a fetal cell, the kit comprising

i) a molecule for detecting a fetal cell, wherein the molecule binds to telomerase, which hybridizes to a polynucleotide encoding a protein component of said telomerase, or which hybridizes to telomeres, and

ii) at least one reagent for detecting said genetic abnormality.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1—Shows a statistics of total numbers of male fetal cells in 10 ml blood samples. Only samples containing male cells are plotted. Fetal cell numbers range from just about 1 cell to more than 100 cells.

FIG. 2—Shows for HLA depletion, the dependence of fetal cell numbers on gestational age.

FIG. 3—Shows fetal cell numbers together with total cell numbers found in the non-retained fraction of the magnetic column.

FIG. 4—Enrichment of fetal cells using combinations of an anti-HLA antibody and an anti-CD45 antibody.

FIG. 5—Data used to produce FIG. 4.

FIG. 6—Effect of auxiliary depletion with CD45 paramagnetic beads.

FIG. 7—Total maternal blood cell contamination after depletion with anti-HLA antibodies+/−CD45 antibodies.

FIG. 8—Comparison between different anti-HLA Class I antibodies.

FIG. 9—Detection of male fetal cells using a RED Y-FISH probe.

FIG. 10—Selection of fetal cells using an anti-telomerase antibody.

### KEY TO THE SEQUENCE LISTING

SEQ ID NO: 1—Human telomerase reverse transcriptase (Genbank Accession No. AAC51724).

SEQ ID NO:2—mRNA encoding human telomerase reverse transcriptase (Genbank Accession No. NM_{—}003219).

SEQ ID NO: 3—RNA component of human telomerase (nucleotides 799 to 1248 of (Genbank Accession No. AF047386).

### DETAILED DESCRIPTION OF THE INVENTION

### General Techniques

Unless specifically defined otherwise, all technical and scientific terms used herein shall be taken to have the same meaning as commonly understood by one of ordinary skill in the art (e.g., in cell culture, fetal cell biology, molecular genetics, immunology, immunohistochemistry, protein chemistry, nucleic acid hybridization, flow cytometry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), and are incorporated herein by reference.

### Major Histocompatibilitv Complex

The major histocompatibility complex (MHC) includes at least three classes of genes. Class I and II genes encode antigens expressed on cell surface, whilst class III genes encode several components of the complement system. Classes I and II antigens are glycoproteins that present peptides to T lymphocytes. Human MHC molecules are also known in the art as Human Leukocyte Antigens (HLA). Thus, the terms “HLA” and “MHC” are often used interchangeably herein.

Human and murine class I molecules are heterodimers, consisting of a heavy alpha chain (45 kD) and a light chain, beta-2-globulin (12 kD). Class I molecules are found on most, if not all, nucleated cells. The alpha chain can be divided into three extracellular domains, alpha1, alpha2 and alpha3, in addition to the transmembranous and cytoplasmic domains. The alpha3 domain is highly conserved, as is beta-2-microglobulin. Both alpha3 domain and beta-2-microglobulin are homologous to the CH3 domain of human immunoglobulin.

Class II molecules are heterodimeric glycoproteins, alpha chain (34 kD) and beta chain (29 kD). Each chain has 2 extracellular domains, together with the transmembranous and cytoplasmic domains. The membrane-proximal alpha2 and beta2 domains are homologous to immunoglobulin CH domain. Class II molecules are less commonly expressed when compared to Class I, typically being found in dendritic cells, B lymphocytes, macrophages, and a few other cell types.

There are 3 class I loci (B,C,A) in the short arm of human chromosome 6, and 4 loci (K, D(L), Qa, Tla) in murine chromosome 17. These loci are highly polymorphic. The variable residues are clustered in 7 subsequences, 3 in alpha1 domain and 4 in alpha2 domain. There are 3 major human class II loci (HLA-DR, HLA-DO, HLA-DP) and 2 murine loci (H-2I-A, H-2I-E). All class II beta chains are polymorphic. Human HLA-DQ alpha chain is also polymorphic.

Preferably, at least some methods of the invention utilize an agent (preferably an antibody) which binds at least one MHC molecule. Preferably, the agent binds an extracellular portion of the MHC molecule. This has at least two advantages, i) the method of the invention can be used to enrich live fetal cells, and ii) an additional step of ensuring that the agent passes through the cell membrane (for example having to fix and permeabilize the cell) is not required.

Preferably, the agent is capable of binding at least one Class I HLA molecule. In one embodiment, the agent is capable of binding HLA-A, HLA-B and HLA-C molecules. In a preferred embodiment, the agent is capable of binding HLA-A and/or HLA-B molecules. In a further embodiment, at least two different agents can be used that bind the same or different Classes or sub-classes of MHC molecules.

As used herein, a “monomorphic determinant” refers to a region of a group proteins that is highly conserved between at least 90%, more preferably at least 95%, more preferably at least 99%, and even more preferably 100% of the group which can be recognised by a suitable binding agent such as an antibody. The region can be a continuous stretch of amino acids, and/or a group of highly conserved amino acids that, upon protein folding, are closely associated. For example, a “monomorphic determinant” of a Class I MHC molecule is a region of the proteins (isotypes) encoded by different alleles of Class I MHC genes that is highly conserved between the different proteins of the Class and that can be bound by the same antibody.

As used herein, a “sub-class” of a MHC molecule is a distinct type of MHC molecules of a particular Class. For example, HLA-A molecules and HLA-B molecules are each considered herein as a sub-class of Class I MHC molecules.

### Telomeres and Telomerases

Telomeres consist of DNA-protein complexes that are located at the ends of eurkaryotic chromosomes and function to provide protection against genome instability promoting events such as degradation of the terminal regions of chromosomes, fusion of a telomere with another telomere or broken DNA end, or inappropriate recombination. Telomeres prior to birth can be considered to be at maximum length. After birth, with each cell division, they get progressively shorter (Vaziri et al., 1994). Telomeric DNA comprises tandem repeats of DNA, in humans the 6-base pair sequence TTAGGG, that form a molecular scaffold containing binding sites for telomeric proteins, resulting in a dynamic DNA-protein complex at the telomere.

Telomerase is an enzyme concerned with the formation, maintenance, and renovation of telomeres at the ends of chromosomes. Telomerase acts as an RNA-dependent DNA polymerase that synthesizes telomeric DNA sequences and consists of two essential components; the first being the functional RNA component (in humans also known as hTR—see SEQ ID NO:3) and the other being the catalytic protein (in humans also known as hTERT—see SEQ ID NO:1). Hence, telomerase is a ribonucleoprotein. Telomerase regulates the proliferative capacity of cells. Telomerase is now classed as a tumour-associated antigen. It may also play a role in the clonal expansion of lymphocytes in response to viral infection.

In biochemical terms, telomerase acts as a telomerase reverse transcriptase (TERT). It transcribes RNA into DNA and is the reverse-transcribing enzyme specific to the telomeric sequence. It has two unique features: it is able to recognize a single-stranded (G-rich) telomere primer and it is able to add multiple telomeric repeats to its end by using its RNA moiety as a template.

The correlation between telomerase activity, telomere lengths, and cellular replicative capacity has led to the theory that maintenance of telomere lengths by telomerase acts as a molecular clock to control replicative capacity and senescence.

The RNA components of human and other telomerases have been cloned and characterized (WO 96/01835). However, the characterization of all the protein components of telomerase has been difficult. Despite this, a number of proteins that may interact with TERT have been identified and include TEP-1 (telomerase associated protein 1) (Harrington et al., 1997) and 14-3-3 proteins (Seimiya et al., 2000).

As used herein, the term “telomerase” refers to at the least the ribonucleoprotein comprising the functional RNA component and the reverse transcriptase. However, at least in some instances this term may also encompass other proteins which may form part of the telomerase complex such as the TEP-1 and 14-3-3 proteins.

### Agent

The present invention relies on the use of various agents which bind molecules expressed by maternal or fetal cells. These agents can be of any structure or composition as long as they are capable binding to a target molecule. In one embodiment, the agents useful for the present invention are proteins. Preferably, the protein is an antibody or fragment thereof.

In an embodiment, it is preferred that an agent is used that binds at least one MHC molecule, and that this agent is an anti-MHC antibody. Preferably, the antibody binds an extracellular portion of the MHC molecule. In another embodiment, the antibody binds specifically to a protein component of telomerase, preferably the reverse transcriptase.

Antibodies useful for the methods of the invention can be monoclonal or polyclonal antibodies. Antibodies useful for the methods of the invention can readily be produced using techniques known in the art. Alternatively, at least some anti-MHC antibodies can be obtained from commercial sources such as US Biological (Massachusetts, USA) and Chemicon International Inc. (California, USA). Furthermore, at least some anti-telomerase antibodies can be obtained from commercial sources such as Abcam Ltd (Cambridge, UK) and Calbiochem (California, USA).

The term “binds specifically” refers to the ability of the antibody to bind to a target ligand (such as telomerase or an MHC molecule) but not other proteins in the sample.

If polyclonal antibodies are desired, a selected mammal (e.g., mouse, rabbit, goat, horse, etc.) is immunised with a suitable immunogenic polypeptide (for example, the extracellular domain of HLA-A can be used when an anti-MHC antibody is desired, or a protein comprising the sequence provided in SEQ ID NO:1 when an anti-telomerase antibody is required). Serum from the immunised animal is collected and treated according to known procedures. If serum containing polyclonal antibodies contains antibodies to other antigens, the polyclonal antibodies can be purified by immunoaffinity chromatography. Techniques for producing and processing polyclonal antisera are known in the art.

Monoclonal antibodies can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced can be screened for various properties; i.e., for isotype and epitope affinity.

An alternative technique involves screening phage display libraries where, for example the phage express single chain antibodies (scFv) fragments on the surface of their coat with a large variety of complementarity determining regions (CDRs). This technique is well known in the art.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as scFv. Furthermore, the antibodies and fragments thereof may be humanised antibodies, for example as described in EP-A-239400.

Preferably, agents used in the methods of the present invention are bound to a detectable label or isolatable label. Alternatively, the agent is not directly labelled but detected using indirect methods such as using a detectably labelled secondary antibody which specifically binds the agent.

The terms “detectable” and “isolatable” label are generally used herein interchangeably. Some labels useful for the methods of the invention cannot readily be visualized (detectable) but nonetheless can be used to enrich (isolate) fetal cells (for example a paramagnetic particle).

Exemplary labels that allow for direct measurement of antibody binding include radiolabels, fluorophores, dyes, magnetic beads, chemiluminescers, colloidal particles, and the like. Examples of labels which permit indirect measurement of binding include enzymes where the substrate may provide for a coloured or fluorescent product. Additional exemplary labels include covalently bound enzymes capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. Further exemplary detectable labels include biotin, which binds with high affinity to avidin or streptavidin; fluorochromes (e.g., phycobiliproteins, phycoerythrin and allophycocyanins; fluorescein and Texas red), which can be used with a fluorescence activated cell sorter; haptens; and the like.

Examples of fluorophores which can be used to label antibodies includes, but are not limited to, Fluorescein Isothiocyanate (FITC), Tetramethyl Rhodamine Isothiocyanate (TRITC), R-Phycoerythrin (R-PE), Alexa™, Dyes, Pacific Blue™, Allophycocyanin (APC), and PerCP™.

The label may also be a quantum dot. In the context of antibody labelling they are used in exactly the same way as fluorescent dyes. Quantum Dots are developed and marketed by several companies, including, Quantum Dot Corporation (USA) and Evident Technologies (USA). Examples of antibodies labelled with quantum dots are described in Michalet et al. (2005) and Tokumasu and Dvorak (2003).

As noted above, in some embodiments the agent is not directly labelled. In this instance, cells are identified using another factor, typically a detectably labeled secondary antibody. The use of detectably labeled secondary antibodies in methods of detecting a marker of interest are well known in the art. For example, if an anti-MHC antibody or anti-telomerase antibody was produced from a rabbit, the secondary antibody could be an anti-rabbit antibody produced from a mouse.

As used herein, the term “sub-saturating concentrations” of an agent such as an antibody means that the number of molecules of the agent is less, preferably significantly less, than the number of target molecules (for example MHC Class I molecules) in a sample. Thus, in this situation only a small fraction of target antigens per cell get an agent bound to them. For example, in some embodiments the ratio of agent to target is less than 1:10, 1:100, 1:1000, or 1:10000. Sub-saturating concentrations of an agent can readily be determined by the skilled person using standard techniques.

Maternal cells bound by an antibody can be killed, and thus depleted from a sample, by complement-dependent lysis. For example, antibody labelled cells can be incubated with rabbit complement at 37° C. for 2 hr. Commercial sources for suitable complement systems include Calbiochem, Equitech-Bio and Pel Freez Biologicals. Suitable anti-MHC antibodies for use in complement-dependent lysis are known in the art, for example the W6/32 antibody mentioned in the Examples can be used for this procedure.

Labelling of Fetal Cells Using a Probe which Binds the RNA Component of Telomerase, the mRNA Encoding a Protein Component of Telomerase, or Telomeres

A probe from use in a method of the invention will typically be DNA, RNA or a mixture thereof. However, the probe may comprise modifications which are usually designed to reduce the likelihood of degradation. Such modifications are typically the use of nucleotide analogs and/or altered linker groups. Nucleic acid analogs which can be used in probes of the invention include phosphoramidate, phosphorothioate, phosphorodithioate, O-methylphosphoroamidite linkages, and peptide nucleic acid backbones and linkages. Other analog nucleic acids include those with positive backbones, non-ionic backbones, and non-ribose backbones. Probes containing one or more carbocyclic sugars are also useful in the methods of the invention.

Preferably a probe used in the methods of the invention is at least 15 nucleotides in length, more preferably at least 20 nucleotides in length, more preferably at least 25 nucleotides in length, more preferably at least 50 nucleotides in length, and even more preferably at least 100 nucleotides in length.

In one embodiment, the probe is capable of hybridizing to a mRNA encoding human TERT (SEQ ID NO:2) or the RNA component of human telomerase (SEQ ID NO:3). The probes of these embodiment are of sufficient length and specificity that there is little, if any, background hybridization to non-target DNA or RNA in the cells of the sample being analysed. Such probes can readily be designed by the skilled person.

In another embodiment, the probe hybridizes to telomeres. As outlined above, human telomeres are repeats of TTAGGG. Thus, probes useful for this embodiment of the invention comprise multiple repeats of this sequence, or the reverse complement thereof. Typically, probes which hybridize telomeres are reasonably long, being at least 1 kb, at least 5 kb, at least 20 kb, at least 50 kb, at least 100 kb, or at least 200 kb in length. Whilst non-fetal cells will also comprise telomeres, fetal cells can still be detected by selecting cells which produce a greater signal upon hybridization with the telomere probe.

Particularly preferred are peptide nucleic acid (PNA) probes which includes peptide nucleic acid analogs. These backbones are substantially non-ionic under neutral conditions, in contrast to the highly charged phosphodiester backbone of naturally occurring nucleic acids. The PNA backbone exhibits improved hybridization kinetics. PNAs have larger changes in the melting temperature (Tm) for mismatched versus perfectly matched basepairs. DNA and RNA typically exhibit a 2-4° C. drop in Tm for an internal mismatch. With the non-ionic PNA backbone, the drop is closer to 7-9. Similarly, due to their non-ionic nature, hybridization of the bases attached to these backbones is relatively insensitive to salt concentration. In addition, PNAs are not degraded by cellular enzymes, and thus can be more stable.

Probes can contain any detection moiety that facilitates the detection of the probe when hybridized to a target nucleic acid sequence (either genomic DNA, mRNA or the RNA component of telomerase). Effective detection moieties include both direct and indirect labels as described below.

Probes can be directly labeled with a detectable label. Examples of detectable labels include, but are not limited to, a fluorescent or chemiluminescent compound, such as fluorescein isothiocyanate, rhodamine, or luciferin, or an enzyme (e.g., as commonly used in an ELISA), biotin, digoxigenin, and radioactive isotopes, e.g., ³²P, and ³H. The detectable label may also be a quantum dot. Fluorophores can be directly labeled following covalent attachment to a nucleotide by incorporating the labeled nucleotide into the probe with standard techniques such as nick translation, random priming, and PCR labeling. Alternatively, nucleotides within the probe can be transaminated with a linker. The fluoropore can then be covalently attached to the transaminated nucleotides. Useful probe labeling techniques are described in Molecular Cytogenetics Protocols and Applications, Y.-S. Fan, Ed., Chap. 2, “Labeling Fluorescence In Situ Hybridization Probes for Genomic Targets”, L. Morrison et. al., p. 21-40, Humana Press, 2002, incorporated herein by reference.

Examples of fluorophores that can be used in the methods described herein include, but are not limited to, 7-amino-4-methylcoumarin-3-acetic acid (AMCA), Texas Red™ (Molecular Probes, Inc., Eugene, Oreg.); 5-(and-6)-carboxy-X-rhodamine, lissamine rhodamine B, 5-(and-6)-carboxyfluorescein; fluorescein-5-isothiocyanate (FITC); 7-diethylaminocoumarin-3-carboxylic acid, tetramethylrhodamine-5-(and-6)-isothiocyanate; 5-(and-6)-carboxytetramethylrhodamine; 7-hydroxycoumarin-3-carboxylic acid; 6-[fluorescein 5-(and-6)-carboxamido]hexanoic acid; N-(4,4-difluoro-5,7-dimethyl-4-bora-3a,4a diaza-3-indacenepropionic acid; cosin-5-isothiocyanate; erythrosine-5-isothiocyanate; 5-(and-6)-carboxyrhodamine 6G; and Cascade™ blue aectylazide (Molecular Probes, Inc., Eugene, Oreg.).

When multiple probes are used, fluorophores of different colours can be chosen such that each probe in a set can be distinctly visualized. For example, activated maternal lymphocytes could be distinguished from fetal cells using such a multiple probe approach.

Probes labeled with a fluorescent moiety can be viewed with a fluorescence microscope and an appropriate filter for each fluorophore, or by using dual or triple band-pass filter sets to observe multiple fluorophores. Any suitable microscopic imaging method can be used to visualize the hybridized probes, including automated digital imaging systems, such as those available from MetaSystems or Applied Imaging. Alternatively, techniques such as flow cytometry can also be used to examine the hybridization pattern of the probes.

Probes can also be labeled indirectly, e.g., with biotin or digoxygenin by means well known in the art. However, secondary detection molecules or further processing are then required to visualize the labeled probes. For example, a probe labeled with biotin can be detected by avidin conjugated to a detectable marker, e.g., a fluorophore. Additionally, avidin can be conjugated to an enzymatic marker such as alkaline phosphatase or horseradish peroxidase. Such enzymatic markers can be detected in standard calorimetric reactions using a substrate for the enzyme. Substrates for alkaline phosphatase include 5-bromo-4-chloro-3-indolylphosphate and nitro blue tetrazolium. Diaminobenzoate can be used as a substrate for horseradish peroxidase.

Digoxigenin PNA probes are available commercially for flow cytometric measurement of telomere length by DAKO Cytomation. Digoxigenin conjugated hybridisations may be detected using anti-digoxigenin fluorescently labelled antibodies. Digoxigenin containing nucleic acid probes can also be produced using a Dig-RNA labelling kit (Roche).

With regard to the detection of telomere length using, for example, a fluorescently labelled PNA probe, a preferred embodiment of the invention is selecting cells that are the most brightly labelled. For instance, in an embodiment fetal cells will typically have a about 1.3 to about 1.5 greater signal than maternal cells. Flow cytometry can be used to measure telomere length (for example, as described by Schmid et al., 2002; Baerlocher et al., 2002; Baerlocher et al., 2003; Cabuy et al., 2004), with analysis algorithms such as those described by De Pauw et al. (1998) and Narath et al. (2005) being suitable to distinguish the more highly labelled fetal cells from the less labelled maternal cells.

### Labelled Fetal Cell Detection and Isolation

As used herein, the terms “enriching” and “enriched” are used in their broadest sense to encompass the isolation of the fetal cells such that the relative concentration of fetal cells to non-fetal cells in the treated sample is greater than a comparable untreated sample. Preferably, the enriched fetal cells are separated from at least 10%, more preferably at least 20%, more preferably at least 30%, more preferably at least 40%, more preferably at least 50%, more preferably at least 60%, more preferably at least 70%, more preferably at least 75%, more preferably at least 80%, more preferably at least 90%, more preferably at least 95%, and even more preferably at least 99% of the non-fetal cells in the sample obtained from the mother. Most preferably, the enriched cell population contains no maternal cells (namely, pure). The terms “enrich” and variations thereof are used interchangeably herein with the term “isolate” and variations thereof. Furthermore, a population of cells enriched using a method of the invention may only comprise a single fetal cell. In addition, the enrichment methods of the invention may be used to isolate a single fetal cell.

Maternal cells expressing at least one type of MHC molecule can be depleted from the sample, by a variety of techniques well known in the art, including cell sorting, especially fluorescence-activated cell sorting (FACS), by using an affinity reagent bound to a substrate (e.g., a plastic surface, as in panning), or by using an affinity reagent bound to a solid phase particle which can be isolated on the basis of the properties of the beads (e.g., colored latex beads or magnetic particles). These same procedures can be used to enrich for cells using telomerase, and/or telomere length, as a marker. Naturally, the procedure used to remove the maternal cells will depend upon how the cells have been labelled.

For removal of maternal cells by cell sorting, the cells are labeled directly or indirectly with a substance which can be detected by a cell sorter, preferably a dye. Preferably, the dye is a fluorescent dye. A large number of different dyes are known in the art, including fluorescein, rhodamine, Texas red, phycoerythrin, and the like. Any detectable substance which has the appropriate characteristics for the cell sorter may be used (e.g., in the case of a fluorescent dye, a dye which can be excited by the sorter's light source, and an emission spectra which can be detected by the cell sorter's detectors). Again, similar techniques can be used to enrich cells using telomerase, and/or telomere length, as a marker.

In flow cytometry, a beam of laser light is projected through a liquid stream that contains cells, or other particles, which when struck by the focussed light give out signals which are picked up by detectors. These signals are then converted for computer storage and data analysis, and can provide information about various cellular properties. Cells labelled with a suitable dye are excited by the laser beam, and emit light at characteristic wavelengths. This emitted light is picked up by detectors, and these analogue signals are converted to digital signals, allowing for their storage, analysis and display.

Many larger flow cytometers are also “cell sorters”, such as fluorescence-activated cell sorters (FACS), and are instruments which have the ability to selectively deposit cells from particular populations into tubes, or other collection vessels. In a particularly preferred embodiment, the cells are isolated using FACS. This procedure is well known in the art and described by, for example, Melamed, et al. (1990) Flow Cytometry and Sorting Wiley-Liss, Inc., New York, N.Y.; Shapiro (2003) Practical Flow Cytometry, 4 ed, Wiley-Liss, Hoboken, N.J.; and Robinson, et al. (1993) Handbook of Flow Cytometry Methods Wiley-Liss, New York, N.Y.

In order to sort cells, the instruments electronics interprets the signals collected for each cell as it is interrogated by the laser beam and compares the signal with sorting criteria set on the computer. If the cell meets the required criteria, an electrical charge is applied to the liquid stream which is being accurately broken into droplets containing the cells. This charge is applied to the stream at the precise moment the cell of interest is about to break off from the stream, then removed when the charged droplet has broken from the stream. As the droplets fall, they pass between two metal plates, which are strongly positively or negatively charged. Charged droplets get drawn towards the metal plate of the opposite polarity, and deposited in the collection vessel, or onto a microscope slide, for further examination.

The cells can automatically be deposited in collection vessels as single cells or as a plurality of cells, e.g. using a laser, e.g. an argon laser (488 nm) and for example with a Flow Cytometer fitted with an Autoclone unit (Coulter EPICS Altra, Beckman-Coulter, Miami, Fla., USA). Other examples of suitable FACS machines useful for the methods of the invention include, but are not limited to, MoFlo™ High-speed cell sorter (Dako-Cytomation ltd), FACS Aria™ (Becton Dickinson), ALTRA™ Hyper sort (Beckman Coulter) and CyFlow™ sorting system (Partec GmbH).

For removal of maternal cells from a sample using solid-phase particles, any particle with the desired properties may be utilized. For example, large particles (e.g., greater than about 90-100 μm in diameter) may be used to facilitate sedimentation. Preferably, the particles are “magnetic particles” (i.e., particles which can be collected using a magnetic field). Typically, maternal cells labelled with the magnetic probe are passed through a column, held within a magnetic field. Labelled cells are retained in the column (held by the magnetic field), whilst unlabelled cells pass straight through and are eluted at the other end. Magnetic particles are now commonly available from a variety of manufacturers including Dynal Biotech (Oslo, Norway) and Milteni Biotech GmbH (Germany). An example of magnetic cell sorting (MACS) is provided by Al-Mufti et al. (1999). Yet again, similar techniques can be used to enrich cells using telomerase, and/or telomere length, as a marker.

Laser-capture microdissection can also be used to selectively remove labelled maternal cells on a slide using methods of the invention. Methods of using laser-capture microdissection are known in the art (see, for example, U.S. 20030227611 and Bauer et al., 2002).

As the skilled person will appreciate, maternal cells can be labelled with one type of label, and fetal cells with another type of label, and the respective cells types identified and/or depleted/selected on the basis of the different labelling. For example, maternal cells can be labelled as described herein such that they produce a fluorescent green signal, and maternal cells can be labelled as described herein such that they produce a fluorescent red signal.

Following enrichment, the cells can be cultured in vitro to expand fetal cells numbers using techniques known in the art. For example culturing in RPMI 1640 media (Gibco).

### Sample and Preparation of Cells

As used herein, the term “sample” refers to material taken directly from the pregnant female (such as blood), as well as such material that has already been partially purified. Examples of such partial purification include the removal of at least some non-cellular material, removal of maternal red blood cells, and/or removal of maternal lymphocytes. Thus, the term “sample” is used herein broadly to include a sample obtained after depletion of maternal cells using, for example, an anti-MHC antibody, but before selection based on the expression of telomerase or telomere length (or vice versa). In some embodiments, the cells in the sample are cultured in vitro before a method of the invention is performed.

The methods of the invention can be performed on any pregnant female of any species, wherein the genome of the species comprises a major histocompatibility complex and/or fetal cells of the organism produce telomerase. Preferably, the female is a mammal. Preferred mammals include, but are not limited to, humans, livestock animals such as sheep, cattle and horses, as well as companion animals such as cats and dogs.

In a preferred embodiment, the sample comprising fetal cells is obtained from a pregnant woman in her first trimester of pregnancy. In one embodiment the sample can be a blood sample which is prevented from clotting such as a sample containing heparin or, preferably, ACD solution. The sample is preferably stored at 0 to 4° C. until use to minimize the number of dead cells, cell debris and cell clumps. The number of fetal cells in the sample varies depending on factors including the age of the fetus. Typically, from 7 to 20 ml of maternal blood provides sufficient fetal cells upon separation from maternal cells. Preferably, 30 ml or more blood is drawn to ensure sufficient cells without the need to draw an additional sample.

In another embodiment, the fetal cells are obtained from the cervical mucous of the mother as, for example, generally described in WO 03/020986, WO 2004/076653 or WO 2005/047532.

In a preferred embodiment, red blood cells are removed from a sample comprising, or derived from, maternal blood. Red blood cells can be removed using any technique known in the art. Red blood cells (erythrocytes) may be depleted by, for example, density gradient centrifugation over Percoll, Ficoll, or other suitable gradients. Red blood cells may also be depleted by selective lysis using commercially available lysing solutions (eg, FACSlyse™, Becton Dickinson), Ammonium Chloride based lysing solutions or other osmotic lysing agents.

Fetal nucleated red cells, if potentially present in the sample, can be protected from ammonium chloride lysis by acetazolamide (Orskoff lysis).

The purity of recovered fetal cells may be increased by depleting the sample of maternal cells using auxiliary agents which bind maternal cell markers other than MHC molecules. The essential feature for choosing such markers for this purpose is that they are not expressed on at least the majority of fetal cells. This auxiliary depletion is performed before, during or after the steps of the invention. Those skilled in the art are aware that the types of nucleated maternal cells in maternal blood include B cells, T cells, monocytes, macrophages dendritic cells and stem cells, each characterised by a specific set of surface markers that can be targeted for depletion. Preferably, the maternal cell population or maternal cells are further depleted by exposing a maternal sample or a nucleated cellular fraction thereof to an antibody that binds to a cellular marker on the maternal cell for a time and under conditions sufficient to form an antibody-maternal cell complex and isolating the antibody-maternal cell complex. As with other embodiments described herein, the antibody-maternal cell complex is preferably isolated by contacting said complex with a readily detectable and/or a readily isolatable label. Examples of non-MHC molecules which can be targeted to possibly further deplete the sample of maternal cells include, but are not limited to, CD3, CD4, CD8, CD10, CD14, CD15, CD45, CD56 and proteins described by Blaschitz et al. (2000). Such further maternal cell specific agents can readily be used in combination with an agent that binds at least one MHC molecule. For example, magnetic beads can be produced which have both anti-MHC and anti-CD45 antibodies attached thereto.

It has been shown that telomerase activity can be detected in cancerous cells (see, for example, Satyanarayana et al., 2004). Thus, when selecting cells for the presence of telomerase or telomere length it is preferred that the sample does not comprise cancerous cells. Such cells can be avoided by screening the individual for cancer before the method of the invention is performed. Such screening can be performed by any method known in the art including analysing the patient, or a sample therefrom, for cancer markers. As the skilled person would be aware, such cancer markers could also be used in methods of removing cancer cells from the sample.

A cancer marker is a molecule which has been shown to be expressed, and/or overexpressed, by a cancer cell. Examples of cancer markers include, but are not limited to, CA 15-3 (marker for numerous cancers including breast cancer), CA 19-9 (marker for numerous cancers including pancreatic cancer and biliary tract tumours), CA 125 (marker for various cancers including ovarian cancer), calcitonin (marker for various tumours including thyroid medullary carcinoma), catecholamines and metabolites (phaeochromoctoma), CEA (marker for various cancers including colorectal cancers and other gastrointestinal cancers), epithelial growth factor (EGF) and/or epithelial growth factor receptor (EGFR) (both associated with colon cancer), A33 colonic epithelial antigen (colon cancer), hCG/beta hCG (marker for various cancers including germ-cell tumours and choriocarcinomas), 5HIAA in urine (carcinoid syndrome), PSA (prostate cancer), sertonin (carcinoid syndrome) NY-ESO-1 (marker of oesophageal cancer), thyroglobulin (thyroid carcinoma), and the CT antigens such as MAGE (associated with many liver cancers and melanomas), GAGE (hepatocarcinoma), SSX2 (sarcoma) differentiation antigens (such as Melan A/MART1, GP100 and tyrosinase), mutational antigens (such as CDK4, β-catenin), amplification antigens (such as P53 and Her2), and splice variant antigens (such as ING1i).

A number of researches have identified that telomerase activity in lymphocytes can lead to false positives when investigating whether a patient has cancer (see, for example, Kavaler et al., 1998; Matthews et al., 2001; Seki et al., 2001; Sidransky, 2002; Trulsson et al., 2003). Accordingly, when selecting cells for the presence of telomerase or telomere length, at least in some circumstances it will be useful to avoid such cells in the sample, and/or take measures to differentially label lymphocytes. Furthermore, when selecting cells for the presence of telomerase or telomere length, it may be useful to ensure the pregnant female does not have an infection which may lead to elevated levels of activated lymphocytes. Lymphocytes can be removed and/or labelled using any technique known in the art. For example, Seki et al. (2001) removed peripheral blood lymphocytes by Ficoll-Isopaque gradient centrifugation before performing a telomerase assay to detect cancer cells. A similar procedure could be used in the present instance. In another example, the cells are pre-sorted by targeting cell surface markers on lymphocytes with a suitable antibody and separating the bound cell. Alternatively, the antibody specific for the lymphocytes could be labeled with a different label than that used to detect the fetal cells, allowing for the two cell types to differentiated as maternal lymphocytes will be doubly labelled whereas the fetal cells will only be labelled with, for example, an antibody which binds telomerase. There are a number of lymphocyte markers which could be used to avoid the false detection of maternal lymphocytes. Suitable T-cell markers include, but are not limited to, CD2, CD3, CD4, CD5, CD6, CD7, CD8, CD25, CD 56, CD94 and CD158a. Suitable B-cell markers include, but are not limited to, CD19 and CD20.

The methods of the invention may include the step of fixing and permeabilizing the cells in the sample. Such procedures are known to those skilled in the art. For example, fixation may involve initial paraformaldehyde fixation followed by treatment with detergents such as Saponin, TWEEN-based detergents, Triton X-100, Nonidet NP40, NP40 substitutes, or other membrane disrupting detergents. Permeabilization may also involve treatment with alcohols (ethanol or methanol). Initial fixation may also be in ethanol. Combined fixation/permeabilization may also be performed using commercially available kits, including DAKO-Intrastain™, Caltag's Fix & Perm reagents, Ortho Diagnostic's Permeafix™.

In other embodiments, such as when electroporation or quantum dots are used to deliver a detectably labelled anti-telomerase antibody to the cells, it is not necessary to fix and permeabilize the cells. As a result, in some embodiments, the methods of the invention can detect and/or isolate live cells. Such isolated live cells could be cultured in vitro to expand fetal cells numbers using techniques known in the art. For example culturing in RPMI 1640 media (Gibco).

Methods for using electroporation to deliver a labelled antibody to a live cell are known in the art (see, for example, Berglund and Starkey, 1989).

### Additional Procedures for the Positive Selection of Fetal Cells

The methods of the invention can include the additional step of positively selecting fetal cells beyond selection based on telomerases or telomere length. Such positive selection relies on targeting molecules produced by fetal cells but not by (or only a small proportion of) the remaining maternal cells. As the skilled person will appreciate, the procedures described above for removing maternal cells expressing at least one MHC molecule are readily adapted for the positive selection of fetal cells expressing a particular cell marker.

For example, fetal cells are selected using cytokeratin-7, a marker on virtually all trophoblast types. Another marker that covers many types of fetal trophoblasts is HLA-G. Further trophoblast-specific antibodies are commercially available, although none of them covers all types of trophoblasts.

In a further example, fetal/embryonic hemoglobin can be used as a marker for fetal nucleated red cells.

Depending on fetal cell types present, such markers can be combined.

### Uses

Enriched fetal cells comprise the same genetic DNA make up of the somatic cells of the fetus, and hence fetal cells isolated using the methods of the invention can be analysed for traits of interest and/or abnormalities using techniques known in the art. Such analysis can be performed on any cellular material that enables the trait, or predisposition thereto, to be detected. Preferably, this material is nuclear DNA, however, at least in some instances it may be informative to analyse RNA or protein from the isolated fetal cells. Furthermore, the DNA may encode a gene, or may encode a functional RNA which is not translated, or the DNA analysed may even be an informative non-transcribed sequence or marker.

In one preferred embodiment, chromosomal abnormalities are detected. By “chromosomal abnormality” we include any gross abnormality in a chromosome or the number of chromosomes. For example, this includes detecting trisomy in chromosome 21 which is indicative of Down's syndrome, trisomy 18, trisomy 13, sex chromosomal abnormalities such as Klinefelter syndrome (47, XXY), XYY or Turner's syndrome, chromosome translocations and deletions, a small proportion of Down's syndrome patients have translocation and chromosomal deletion syndromes include Pradar-Willi syndrome and Angelman syndrome, both of which involve deletions of part of chromosome 15, and the detection of mutations (such as deletions, insertions, transitions, transversions and other mutations) in individual genes. Other types of chromosomal problems also exist such as Fragile X syndrome, hemophilia, spinal muscular dystrophy, myotonic dystophy, Menkes disease and neurofibromatosis, which can be detected by DNA analysis.

The phrase “genetic abnormality” also refers to a single nucleotide substitution, deletion, insertion, micro-deletion, micro-insertion, short deletion, short insertion, multinucleotide substitution, and abnormal DNA methylation and loss of imprint (LOI). Such a genetic abnormality can be related to an inherited genetic disease such as a single-gene disorder (e.g., cystic fibrosis, Canavan, Tay-Sachs disease, Gaucher disease, Familial Dysautonomia, Niemann-Pick disease, Fanconi anemia, Ataxia telaugiestasia, Bloom syndrome, Familial Mediterranean fever (FMF), X-linked spondyloepiphyseal dysplasia tarda, factor XI), an imprinting disorder [e.g., Angelman Syndrome, Prader-Willi Syndrome, Beckwith-Wiedemann syndrome, Myoclonus-dystonia syndrome (MDS)], or to predisposition to various diseases (e.g., mutations in the BRCA1 and BRCA2 genes). Other genetic disorders which can be detected by DNA analysis are known such as thalassaemia, Duchenne muscular dystrophy, connexin 26, congenital adrenal hypoplasia, X-linked hydrocephalus, ornithine transcarbamlyase deficiency, Huntington's disease, mitochondrial disorder, mucopolysaccharidosis I or IV, Norrie's disease, Rett syndrome, Smith-Lemli Optiz syndrome, 21-hydroxylase deficiency or holocarboxylase synthetase deficiency, diastrophic displasia, galactosialidosis, gangliosidosis, hereditary sensory neuropathy, hypogammaglobulinaemia, hypophosphatasia, Leigh's syndrome, aspartylglucosaminuria, metachromatic leukodystrophy Wilson's disease, steroid sulfatase deficiency, X-linked adrenoleukodystrophy, phosphorylase kinase deficiency (Type VI glycogen storage disease) and debranching enzyme deficiency (Type III glycogen storage disease). These and other genetic diseases are mentioned in The Metabolic and Molecular Basis of Inherited Disease, 8th Edition, Volumes I, II, III and IV, Scriver, C. R. et al. (eds), McGraw Hill, 2001. Clearly, any genetic disease where the gene has been cloned and mutations detected can be analysed.

The methods of the present invention can also be used to determine the sex of the fetus. For example, staining of the isolated fetal cells with a Y-chromosome specific marker will indicate that the fetus is male, whereas the lack of staining will indicate that the fetus is female.

In yet another use of the invention, the methods described herein can be used for paternity testing. Where the paternity of a child is disputed, the procedures of the invention enable this issue to be resolved early on during pregnancy. Many procedures have been described for parentage testing which rely on the analysis of suitable polymorphic markers. As used herein, the phrase “polymorphic markers” refers to any nucleic acid change (e.g., substitution, deletion, insertion, inversion), variable number of tandem repeats (VNTR), short tandem repeats (STR), minisatellite variant repeats (MVR) and the like. Typically, parentage testing involves DNA fingerprinting targeting informative repeat regions, or the analysis of highly polymorphic regions of the genome such as HLA loci.

### Analysis of Fetal Cells

Fetal cells enriched/detected using the methods of the invention can be analysed by a variety of procedures, however, typically genetic assays will be performed. Genetic assay methods include the standard techniques of karyotyping, analysis of methylation patterns, restriction fragment length polymorphism assays, sequencing and PCR-based assays, as well as other methods described below.

Chromosomal abnormalities, either in structure or number, can be detected by karyotyping which is well known in the art. Karyotyping analysis is generally performed on cells which have been arrested during mitosis by the addition of a mitotic spindle inhibitor such as colchicine. Preferably, a Giemsa-stained chromosome spread is prepared, allowing analysis of chromosome number as well as detection of chromosomal translocations.

The genetic assays may involve any suitable method for identifying mutations or polymorphisms, such as: sequencing of the DNA at one or more of the relevant positions; differential hybridisation of an oligonucleotide probe designed to hybridise at the relevant positions of either the wild-type or mutant sequence; denaturing gel electrophoresis following digestion with an appropriate restriction enzyme, preferably following amplification of the relevant DNA regions; S1 nuclease sequence analysis; non-denaturing gel electrophoresis, preferably following amplification of the relevant DNA regions; conventional RFLP (restriction fragment length polymorphism) assays; selective DNA amplification using oligonucleotides which are matched for the wild-type sequence and unmatched for the mutant sequence or vice versa; or the selective introduction of a restriction site using a PCR (or similar) primer matched for the wild-type or mutant genotype, followed by a restriction digest. The assay may be indirect, ie capable of detecting a mutation at another position or gene which is known to be linked to one or more of the mutant positions. The probes and primers may be fragments of DNA isolated from nature or may be synthetic.

A non-denaturing gel may be used to detect differing lengths of fragments resulting from digestion with an appropriate restriction enzyme. The DNA is usually amplified before digestion, for example using the polymerase chain reaction (PCR) method and modifications thereof.

Amplification of DNA may be achieved by the established PCR methods or by developments thereof or alternatives such as the ligase chain reaction, QB replicase and nucleic acid sequence-based amplification.

An “appropriate restriction enzyme” is one which will recognise and cut the wild-type sequence and not the mutated sequence or vice versa. The sequence which is recognised and cut by the restriction enzyme (or not, as the case may be) can be present as a consequence of the mutation or it can be introduced into the normal or mutant allele using mismatched oligonucleotides in the PCR reaction. It is convenient if the enzyme cuts DNA only infrequently, in other words if it recognises a sequence which occurs only rarely.

In another method, a pair of PCR primers are used which hybridise to either the wild-type genotype or the mutant genotype but not both. Whether amplified DNA is produced will then indicate the wild-type or mutant genotype (and hence phenotype).

A preferable method employs similar PCR primers but, as well as hybridising to only one of the wild-type or mutant sequences, they introduce a restriction site which is not otherwise there in either the wild-type or mutant sequences.

In order to facilitate subsequent cloning of amplified sequences, primers may have restriction enzyme sites appended to their 5′ ends. Thus, all nucleotides of the primers are derived from the gene sequence of interest or sequences adjacent to that gene except the few nucleotides necessary to form a restriction enzyme site. Such enzymes and sites are well known in the art. The primers themselves can be synthesized using techniques which are well known in the art. Generally, the primers can be made using synthesizing machines which are commercially available.

PCR techniques that utilize fluorescent dyes may also be used to detect genetic defects in DNA from fetal cells isolated by the methods of the invention. These include, but are not limited to, the following five techniques.

i) Fluorescent dyes can be used to detect specific PCR amplified double stranded DNA product (e.g. ethidium bromide, or SYBR Green I).

ii) The 5′ nuclease (TaqMan) assay can be used which utilizes a specially constructed primer whose fluorescence is quenched until it is released by the nuclease activity of the Taq DNA polymerase during extension of the PCR product.

iii) Assays based on Molecular Beacon technology can be used which rely on a specially constructed oligonucleotide that when self-hybridized quenches fluorescence (fluorescent dye and quencher molecule are adjacent). Upon hybridization to a specific amplified PCR product, fluorescence is increased due to separation of the quencher from the fluorescent molecule.

iv) Assays based on Amplifluor (Intergen) technology can be used which utilize specially prepared primers, where again fluorescence is quenched due to self-hybridization. In this case, fluorescence is released during PCR amplification by extension through the primer sequence, which results in the separation of fluorescent and quencher molecules.

v) Assays that rely on an increase in fluorescence resonance energy transfer can be used which utilize two specially designed adjacent primers, which have different fluorochromes on their ends. When these primers anneal to a specific PCR amplified product, the two fluorochromes are brought together. The excitation of one fluorochrome results in an increase in fluorescence of the other fluorochrome.

If required, methods for the extraction of DNA from fixed samples for genetic analysis are also known to those skilled in the art. For example, US patent application 20040126796 discloses a method for the extraction of DNA from tissues and other samples, such as formalin-fixed tissue. The isolation of DNA from fixed samples for use in PCR has also been described by Lehman and Kreipe (2001) and Fitzgerald et al. (1993).

Fetal cells, or an enriched cell population of fetal cells, obtained using a method of the invention can be placed into wells of a microtitre plate (one cell per well) and analysed independently. Preferably, each cell not only screened for a trait(s) of interest, but screened to confirm/detect that the cell in a particular well is a fetal cell. In this instance, multiplex analysis can be performed as generally described by Finlay et al. (1996, 1998 and 2001).

### Kits

The present invention also provides a kits for enriching fetal cells from a sample. In one example, the kit comprises i) an agent that binds at least one MHC molecule, an agent that binds a compound that associates with an MHC molecule, and/or an agent that binds a hemopoietic cell, and ii) a molecule which binds to telomerase, and/or which hybridizes to a polynucleotide encoding a protein component of said telomerase, and/or which hybridizes to telomeres. Other examples are described herein.

In one embodiment, a kit of the present invention includes, a single agent in an amount sufficient for at least one enrichment and/or detection procedure. Kits containing multiple agents are also contemplated by the present invention. The multiple agents may bind different MHC molecules of the same Class, and/or bind unrelated molecules (such as one agent that binds a monomorphic determinant of HLA-A molecules and another agent that binds CD45). Such agents may be bound to detectable or isolatable labels. For ease of use, multiple agents are typically bound to the same detectable or isolatable label.

In one embodiment, the agent(s) are each linked to magnetic beads. Different agents may be linked to different beads such that a single type of bead comprises different types of agents, or beads may be produced that only comprises a single type of agent and these beads mixed with other beads that have linked thereto a single type, but different, agent.

The kit may further comprise components for analysing the genotype of a fetal cell, determining the father of a fetus, and/or determining the sex of the fetus.

Typically, the kits will also include instructions recorded in a tangible form (e.g., contained on paper or an electronic medium), for example, for using a packaged agent for enriching fetal cells from a sample. The instructions will typically indicate the reagents and/or concentrations of reagents and at least one enrichment method parameter which might be, for example, the relative amounts of agents to use per amount of sample. In addition, such specifics as maintenance, time periods, temperature and buffer conditions may also be included.

### EXAMPLES

### Example 1

### Enrichment of Fetal Cells Using Mouse Anti-Human HLA Class 1 Antigen Antibodies

### Materials and Methods

### Blood

Blood samples were obtained from a private abortion clinic and the Royal Children's Hospital (RCH) (Melbourne, Australia). Sample collection was anonymous, with donors de-identified. Whilst samples from the abortion clinic were specified to be pre-abortive, it was later determined that some of the samples that yielded higher numbers of fetal cells had been obtained post-abortion.

Blood samples (8-16 ml) were drawn into vacuum collection tubes with EDTA as anti-coagulant. The samples were processed either fresh or after overnight storage at 4° C.

### Magnetic Cell Separation

Mononuclear cells were isolated by density gradient (Ficoll 1.077) centrifugation, and the entire samples were magnetically labelled with either of the following three procedures:
1. Cells were exposed to saturating amounts of a biotinylated antibody against a HLA Class 1 epitope common to all HLA-A, B and C (US Biological; Cat# H6098-39F2; Mouse anti-Human HLA Class 1 Antigen ABC/Biotin; IgG2a; Clone 3H2211). Cells were then washed and labelled with saturating amounts of streptavidin-coated paramagnetic particles (Molecular Probes/Invitrogen; Cat# C-21476 “Captivate”).
2. Cells were exposed to saturating amounts of paramagnetic particles coated with antibodies to CD45 (Miltenyi Cat# 130-090-872; concentrated “whole-blood” anti-human CD45 beads).
3. Procedures 1 and 2 were combined: Cells were first labelled to the HLA antibody, followed with a simultaneous exposure to Captivate and CD45 magnetic beads.

Magnetically labelled cell samples were passed through a magnetised column (Miltenyi, LS columns Cat# 130-042-401), retaining all labelled cells. The non-adhered as well as the adhered fractions were collected, pelleted and frozen at −80° C. until further use.

### Detection of Fetal Cells by Quantitative PCR (Q-PCR)

DNA was prepared using a commercial kit (Qiagen Cat# 51204 “FlexiGene DNA kit), and Q-PCR was performed on a real-time PCR machine (StrataGene MX3000), targeting a Y-chromosome-specific multi-copy sequence. Parallel reactions targeting a gender-unspecific sequence were performed to quantitate the total amount of DNA in the sample. All PCR reagents were from Qiagen. By comparison with standards derived from known amounts of pure male-derived DNA, the total numbers of male (=fetal) cells and all cells in the magnetically separated preparations were calculated.

### Results and Discussion

Among 30 samples processed with HLA Class 1 cell depletion, 14 samples contained male cells. Since about half of all fetuses are female, a nearly 50% detection rate of male cells indicates that fetal cells are retrieved in nearly every maternal sample.

Among 9 samples processed with CD45 cell depletion, 5 samples contained male cells. Again, this is a approximately 50% detection rate and indicates that fetal cells are always retrieved.

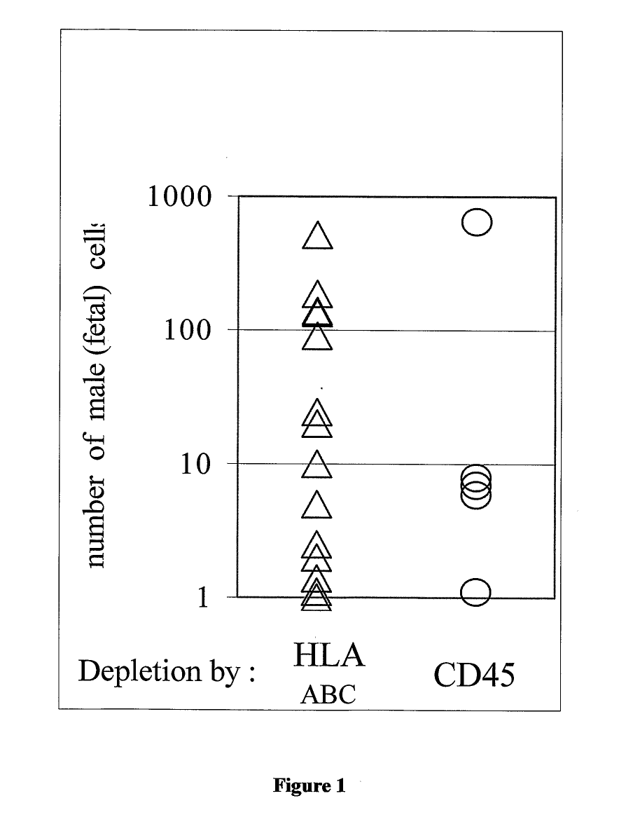

Provided in FIG. 1 are statistics of total numbers of male fetal cells in 10 ml blood samples. Only samples containing male cells are plotted. Fetal cell numbers range from just about 1 cell to more than 100 cells.

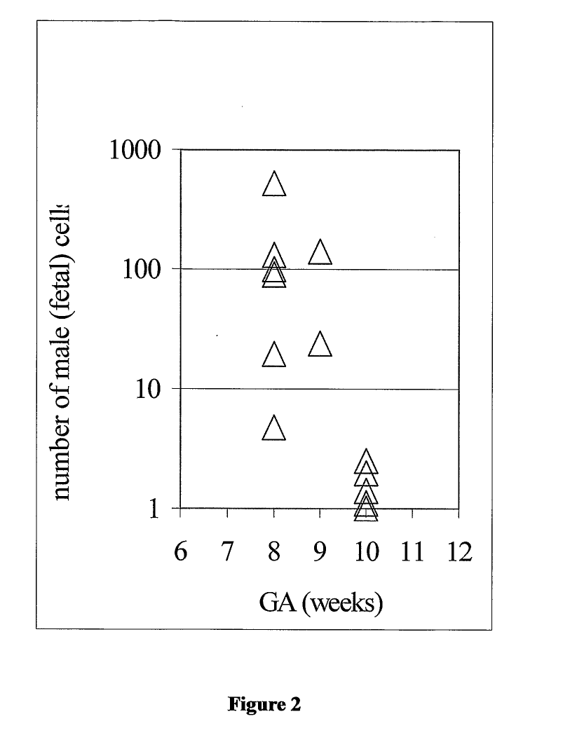

FIG. 2 shows, for HLA depletion, the dependence of fetal cell numbers on gestational age.

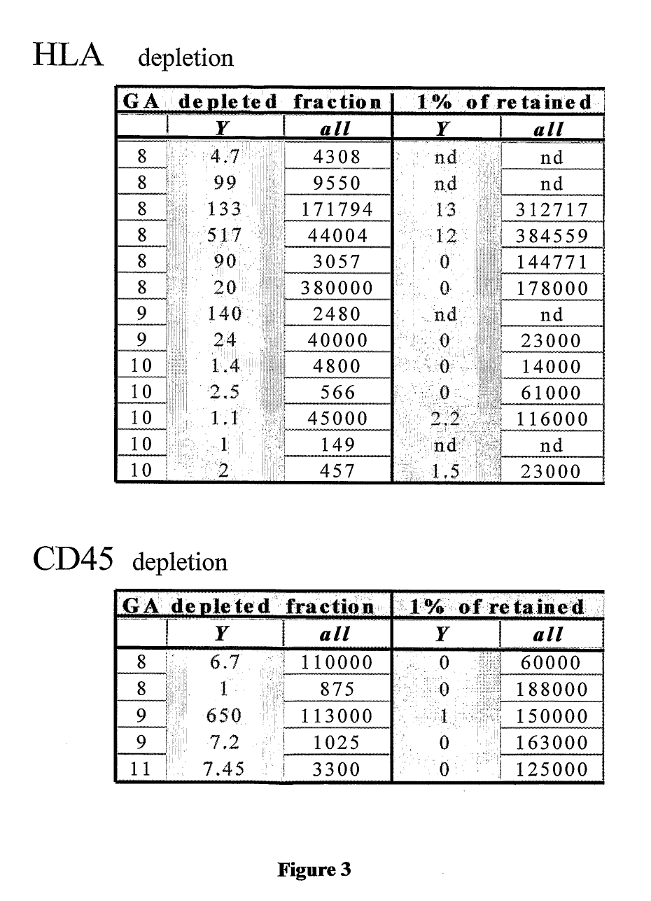

FIG. 3 provides fetal cell numbers together with total cell numbers found in the non-retained fraction of the magnetic column. The numbers vary from less than 1000 to about 100,000. With an approximate 10 million cells in the starting population of mononuclear cells, this is a dramatic enrichment. Also shown are the controls in which 1% of the retained cell fraction was examined for fetal cells. The presence of some occasional fetal cells in 1% of the controls indicates that not all fetal cells are retrieved with these procedures, but that some are CD45+ and HLA C1.1+.

### Example 2

### Enrichment of Fetal Cells Using Mouse Anti-Human HLA Class 1 Antigen ABC Clone 39-F2 or Clone W6/32, Both in Combination with an Anti-CD45 Antibody

Unless stated to the contrary the procedures used were the same as those described above for Example 1.

Blood at different gestational ages was subjected to gradient centrifugation, removing erythrocytes, then labelled with either of two clones (39-F2 and W6/32) of biotinylated monoclonal antibody, each directed against a different monomorphic determinant of HLA-A,B,C. [US Biological, USA]. Subsequently, the cells were labelled simultaneously with streptavidin-coated paramagnetic beads (“Captivate”, Moleculare Probes, USA] and paramagnetic beads coated with an antibody against the CD45 antigen [Miltenyi, Germany].

The labelled cells were passed through a magnetic column [Miltenyi], and the non-attached cells were subjected to quantitative PCR, targeting a Y-chromosome-specific sequence.

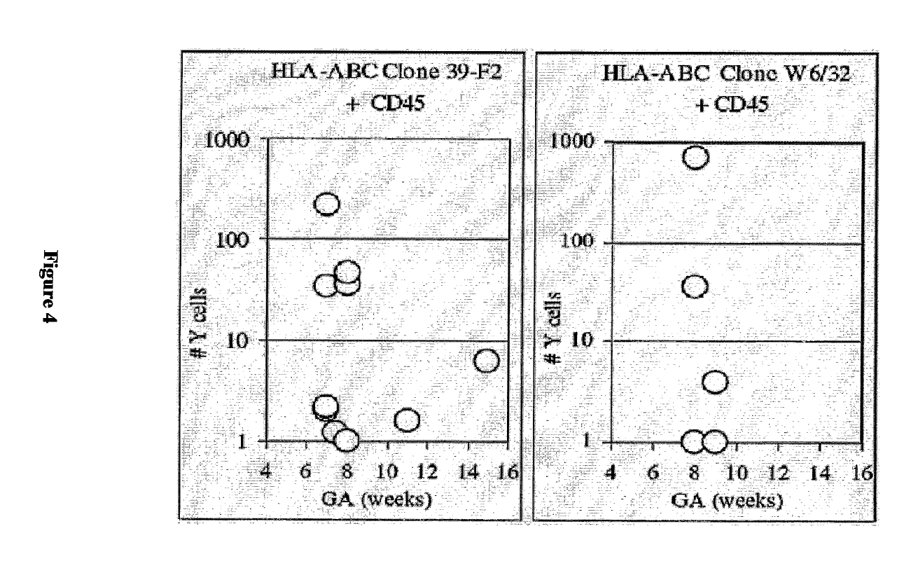

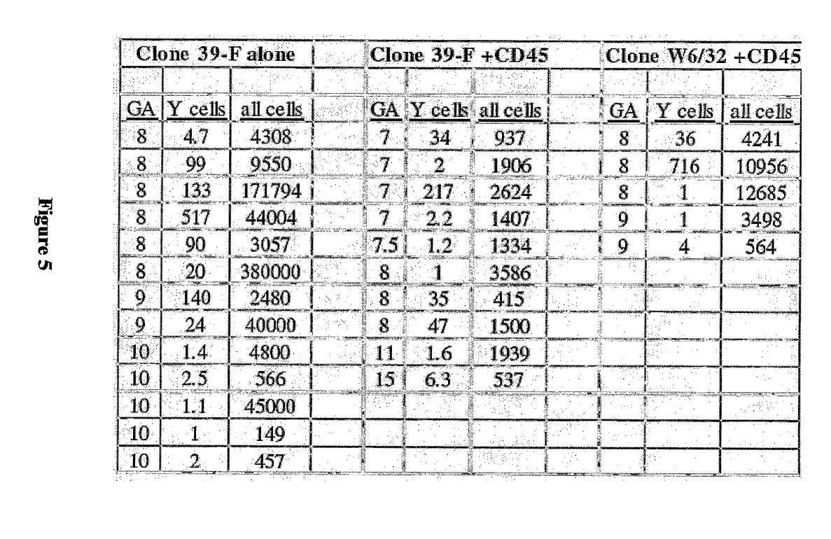

FIGS. 4 and 5 show total fetal (male) cell numbers per 10 ml of blood, plotted as a function of gestational age (GA). Nearly half of all blood samples (with unknown fetal gender) yielded a Y-signal. Only the positive samples (with at least one male cell) are shown.

These results complement those provided in Example 1. They provide the following additional information:
a. The fetal cell enrichment achieved by this method does not depend on a special HLA-ABC epitope targeted by one particular HLA-ABC antibody clone, but can be achieved with different antibodies targeting different epitopes on the Class 1 antigens.
b. An enhanced enrichment of fetal cells is obtained using a combination of antibodies that bind MHC molecules and hemopoietic cells (in this case an anti-CD45 antibody) (FIG. 5).
c. Similar numbers of fetal cells are found at gestational ages 7 and 8 weeks, and even as early as week 6 there are fetal cells to be found.

### Example 3

### Further Studies Showing Enrichment of Fetal Cells Using Mouse Anti-Human HLA Class 1 Antigen ABC Antibodies

### Materials and Methods

### Blood

Blood of pregnant women was obtained from a private abortion clinic and the Royal Children's Hospital (RCH) (Melbourne, Australia). Maternal blood samples were drawn in steady-state pregnancy, prior to any testing or abortive procedure that could release fetal cells into the maternal circulation. For use as a model system, other samples were drawn during or after termination of pregnancy (post-termination samples) to provide blood samples with increased numbers of fetal cells due to fetal hemorrhage. Sample collection was anonymous, with donors de-identified.

Blood samples (8-16 ml) were drawn into vacuum collection tubes with EDTA as anti-coagulant. The samples were processed either fresh or after overnight storage at 4° C.

### Magnetic Cell Separation

Mononuclear cells were isolated by density gradient (Ficoll 1.083) centrifugation, and the entire samples were magnetically labelled with either of the following three procedures:
1. Cells were exposed to saturating amounts of one of the following biotinylated antibodies against a HLA Class 1 epitope common to all HLA-A, B and C:
   a. US Biological; Cat # H6098-39F2; Mouse anti-Human HLA Class 1 Antigen ABC; (Data code: F2)
   b. USBiological; Cat# H6098-60B; Mouse anti-Human HLA Class 1 Antigen ABC (Data code: 60B)
   c. EBioscience; Cat# 13-9983-82; Mouse anti-Human HLA Class 1 Antigen ABC; clone W6/32 (Data code: W6), or
   antibodies against epitopes on HLA-B locus: one Lambda; mouse anti-human Bw4 (cat #BIH0007; mouse anti-human IgG2a); mouse anti-human Bw6 (cat #BIH0038; mouse anti-human IgG3) (Data code Bw4/6).
   Cells were then washed and labelled with saturating amounts of streptavidin-coated paramagnetic particles (Molecular Probes/Invitrogen; Cat# C-21476 “Captivate”).
2. Cells were exposed to saturating amounts of paramagnetic particles coated with antibodies to CD45 (Miltenyi Cat# 130-090-872; concentrated “whole-blood” anti-human CD45 beads).
3. Procedures 1 and 2 were combined: Cells were first labelled to the HLA antibody, followed with a simultaneous exposure to Captivate and CD45 magnetic beads.

Magnetically labelled cell samples were passed through a magnetised column (Miltenyi, LS columns Cat# 130-042-401), retaining all labelled cells. The non-retained as well as the retained fractions were collected, pelleted and frozen at −80° C. until further use.

### Detection of Fetal Cells by Quantitative PCR (Q-PCR)

DNA was prepared using a commercial kit (Qiagen Cat# 51204 “FlexiGene DNA kit), and Q-PCR was performed on a real-time PCR machine (StrataGene MX3000), targeting a Y-chromosome-specific multi-copy sequence. Parallel reactions targeting a gender-unspecific sequence were performed to quantitate the total amount of DNA in the sample. All PCR reagents were from Qiagen. By comparison with standards derived from known amounts of pure male-derived DNA, the total numbers of male (=fetal) cells and all cells in the magnetically separated preparations were estimated. At low fetal cell numbers (<10), this method was found to under-estimate fetal cell numbers.

### Results and Discussion

Table 1 shows the fetal cell detection rates in steady-state maternal blood samples collected between week 7 and 14 of pregnancy. 101 samples were processed with HLA Class 1 and CD45 cell depletion. As many as 43 samples produced a clear Y-chromosome-specific signal, indicating that they contained at least 1 fetal cell. Since about half of all fetuses are female, a nearly 50% detection rate of male cells indicates that fetal cells are retrieved in nearly every maternal sample. Considering that the PCR method was found to under-estimate fetal cell numbers in the range from 1-10, we suggest that the true fetal cell recovery is higher than the detection rate, probably 100%.

TABLE 1
Detection of fetal cells in steady-state, first trimester maternal blood.

| GA | samples | Y signal | |
|---|---|---|---|
| 7 | 17 | 7 | |
| 8 | 46 | 17 | |
| 9 | 9 | 6 | |
| 10 | 17 | 6 | |
| 12 | 7 | 5 | |
| 11 | 2 | 1 | |
| 13 | 2 | 0 | |
| 14 | 1 | 1 | |
| total | 101 | 43 | 42.60% |

The data indicates that fetal cells can be found as early as 7 weeks GA, a result that appears to be a dramatic improvement over any other published results.

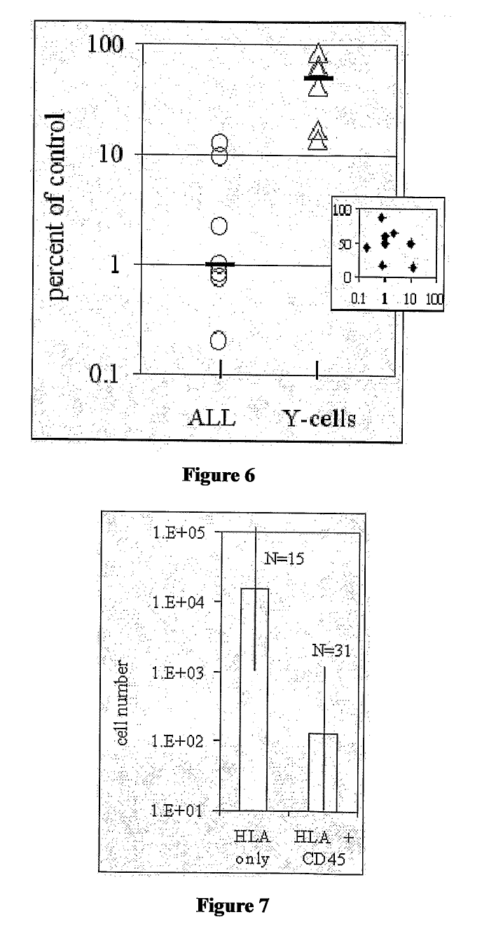

Similar to Example 2, FIG. 6 shows the effect of the auxiliary use of CD45 depletion in addition to cell depletion with HLA Class I antibody, using post-termination blood samples, which serve as a model system with increased numbers of fetal cells. Nucleated blood cells were incubated with biotinylated antibody to HLA Class I antigen (Bw4+6), followed by incubation with streptavidin ferrofluid. Half of the sample was simultaneously incubated with paramagnetic beads binding to CD45 antigen, the other half served as control. Total numbers of remaining cells, as well as the numbers of male cells, were determined by Q-PCR. The ratios of cell numbers after HLA+CD45 depletion were divided by cell numbers after only HLA depletion and are shown as % of control. The plot of ALL vs. Y values for each sample (insert graph of FIG. 6) shows the lack of correlation between the two values. The graph shows that the auxiliary depletion by CD45 beads reduced the total remaining cell numbers to 1 percent of controls (HLA depletion only), while the numbers of fetal cells are only reduced by about 50%.

In a further experiment, maternal blood (10-12 ml) was processed by density gradient (1.083), nucleated cells were labelled with anti-HLA Bw4+Bw6/biotin, then depleted with streptavidin ferrofluid+/−anti-CD45 paramagnetic beads. Total remaining cell numbers (mostly maternal, of course) were determined by Q-PCR. The results are provided in FIG. 7 with median values and approximate range being shown. The data show that depletion with HLA+CD45 results in total cell numbers of about 100 cells on average, which implies that average fetal cell purity is >1%, whenever any number of fetal cells are present (FIG. 7). These relatively few contaminating maternal cells and resulting high fetal cell purity will enable positive fetal cell markers to positively identify fetal cells by microscopy or single-cell PCR techniques.

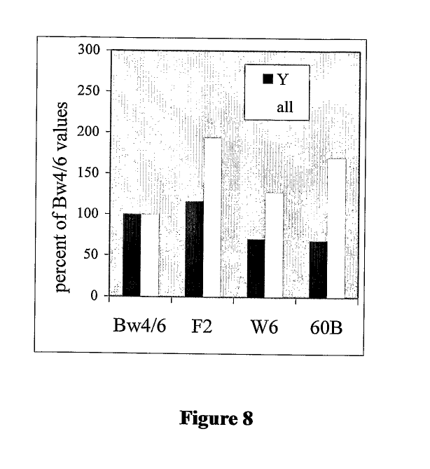

FIG. 8 provides a comparison between different HLA-Class I antibodies with respect to fetal cell recovery and total cell depletion. Three of the antibodies (F2, 60B and W6/32) are directed to 3 different epitopes common to all HLA-A, B and C antigens. Bw4/6 is a mixture of specific antibodies to Bw4 and Bw6. A person is Bw4, Bw6 or both, so that the combination of both ensures antibody binding for each blood donor. The data show that there is little difference between the different antibodies, which implies a wide choice of commercially available antibodies for this method.

### Example 4

### Detection of Human Telomerase Reverse Transcriptase Protein (hTERT) by Monoclonal or Polyclonal Antibodies

Two protocols are provided below for the detection of human telomerase reverse transcriptase protein (hTERT) by monoclonal or polyclonal antibodies. As the skilled person would be aware, many of individual procedures described below are interchangeable between the two protocols.

### Protocol 1

Cells from blood of a pregnant female are separated from plasma by centrifugation. Red cells are depleted on Percoll density gradients. Cells are fixed and permeabilized using a commercial kit—DAKO-Intrastain. The cells are washed again in PBS, and then incubated with monoclonal anti-telomerase antibody (Abcam Ltd, Cambridge, UK) for 1 hour at room temperature.

The cells are then washed in PBS (150 mM NaCl, 10 mM phosphate buffer) containing 0.5% bovine serum albumin (BSA), and a Fluorescein Isothiocyanate (FITC) fluorescently labelled secondary antibody which binds the monoclonal antibody is added for 1 hour at room temperature. Cells are washed in PBS containing 0.5% BSA.

Cells are analysed and labelled cells separated using fluorescence activated cell sorting on a MoFlo High-speed cell sorter (Dako-Cytomation, Ltd).

### Protocol 2

Cells from blood of a pregnant female are separated from plasma by centrifugation. Red cells are depleted by selective lysis using Becton Dickinson FACSLyse solution. Cells are fixed in paraformaldehyde (about 1.5%) for 24 hours at 4° C. Cells are washed in PBS and permeabilised using 0.05% Triton X-100 in PBS for 30 min at room temp.

The cells are washed again in PBS, and then incubated with a polyclonal antisera comprising anti-telomerase antibodies (Calbiochem, California, USA) which are labelled with magnetic beads (Dynal Biotech) for 1 hour at room temperature.

The cells are then washed in PBS containing 0.5% bovine serum albumin (BSA), and analysed and labelled cells separated using magnetic activated cell sorting.

### Example 5

### Detection of hTERT mRNA by Hybridisation

Cells from blood of a pregnant female are separated from plasma by centrifugation. Red cells are depleted on 70% Percoll density gradients. Cells are fixed and permeabilized using a commercial kit—Caltag Fix & Perm.

The cell suspension is centrifuged (1000 g, 5 min), and the cells resuspended in 500 μl ice-cold methanol and incubated for 10 min at 4° C. The cells are centrifuged at 1000 g for 5 min, and resuspended in 500 μl 0.2% Triton X-100/TE buffer (TE=Tris/EDTA buffer (10 mM Tris/1 mM EDTA pH 7.2). The cells are centrifuged again at 1000 g for 5 min, and the supernatant carefully removed. Cells are washed once in 500 μl TE and centrifuged at 1000 g for 5 min. Cells are resuspended in 5 μl of TE (avoiding bubbles).

20 μl of riboprobe comprising fluorescein-UTP is added in hybridization buffer (50% Formamide, 10 mM Tris (pH 7.0), 5 mM EDTA, 10% Dextran Sulphate, 1 μg/μl tRNA). The riboprobe has a sequence which is complementary to the mRNA encoding hTERT, and is produced using techniques known in the art (Sambrook et al., supra). The hybridization proceeds for 12 hours at 45° C. Cells are washed with 2×SSC buffer, and pelleted at 1000 g for 5 min. As much supernatant as possible is removed, and the cells resuspended in 200 μl 2×SSC/0.3% NP40.

The cells are incubated at 37° C. for 30 min. The cells are then centrifuged at 1000 g 5 min, and the supernatant carefully removed. The cells are then resuspended in 200 μl 2×SSC/0.3% NP40 and incubated at room temp for 30 min. The cells are then centrifuged at 1000 g 5 min, and the supernatant carefully removed. The cells are then resuspended in 2.5 μl of TE.

Cells are analysed and labelled cells separated using fluorescence activated cell sorting on a MoFlo High-speed cell sorter (Dako-Cytomation, Ltd).

### Example 6

### Determination of Telomere Length with a PNA Hybridisation Probe

Cells from blood are separated from plasma by centrifugation. Red cells are depleted on Percoll density gradients. Cells are fixed and permeabilized using a commercial kit—Caltag Fix & Perm. 100 μl of the resulting fixed cells are placed in an eppendorf tube and centrifuged (1000g, 5 min).

Cells are resuspended in 500 μl ice-cold methanol and incubated for 10 min at 4° C., and the centrifuged at 1000 g for 5 min. The cells are resuspended in 500 μl 0.2% Triton X-100/TE buffer, centrifuge at 1000 g for 5 min, and then the supernatant carefully removed.

Cells are washed once in 500 μl TE and centrifuged at 1000 g for 5 min. Cells are resuspended in 5 μl of TE (avoiding bubbles). 20 μl of PNA (Dako Telomere PNA kit/FITC, Dako-Cytomation) in hybridization buffer is added and co-denatured at 80° C. for 20 min in thermocycler.

Hybridization is allowed to proceed for 12 hours at 37° C. The cells are then washed with 2×SSC buffer, and pelleted at 1000 g for 5 min. As much supernatant as possible is removed, and the cells resuspended in 200 μl 2×SSC/0.3% NP40. The cells are incubated at 37° C. for 30 min, centrifuged at 1000 g for 5 min, and as much supernatant as possible removed. The cells are then resuspended in 200 μl 2×SSC/0.3% NP40, and incubated at room temp for 30 min. The cells are then centrifuged at 1000 g for 5 min. As much supernatant as possible is removed and the cells resuspended in 2.5 μl TE.

Cells are analysed and labelled cells separated using fluorescence activated cell sorting on a MoFlo High-speed cell sorter (Dako-Cytomation, Ltd).

### Example 7

### Labelling Fetal Cells Using Anti-Telomerase Antibody

10 ml samples of peripheral blood were obtained by venupuncture from female volunteers during the first trimester of pregnancy.

Red cells were depleted by density gradient centrifugation over a gradient of 70% Percoll. The collected cells were washed in PBS containing 5% BSA and then fixed overnight in 2% paraformaldehyde at 4° C.

Cells were washed in PBS then permeabilized using 0.05% Triton X-100 (in PBS) for 30 min at 4° C. Cells were washed once in PBS and resuspended in 500 μl PBS. Anti-Telomerase polyclonal antibody (Abcam Ltd, Cambridge, UK) (10 μg contained in 100 μl H₂O) was added to the cells and incubated at 4° C. for 4 hours. Cells were washed twice in PBS and resuspended in 500 μl PBS. 100 μl Goat-anti-rabbit IgG-FITC was added and the cells incubated for 1 hour at 4° C. Cells were washed twice in PBS and resuspended in 5 ml PBS.

Cells were then analysed and sorted using a Dako-Cytomation MoFlo high speed cell sorter. Sort gates were set on cells expressing the top 5% of fluorescence values for this initial experiment.

Male fetal cells are labelled with RED (Spectrum Orange™) Y-FISH probe (Vysis, USA) and Green (Spectrum Green™) X-FISH probe (Vysis, USA). Male fetal cells are those which express 1 Red and 1 Green FISH signal.

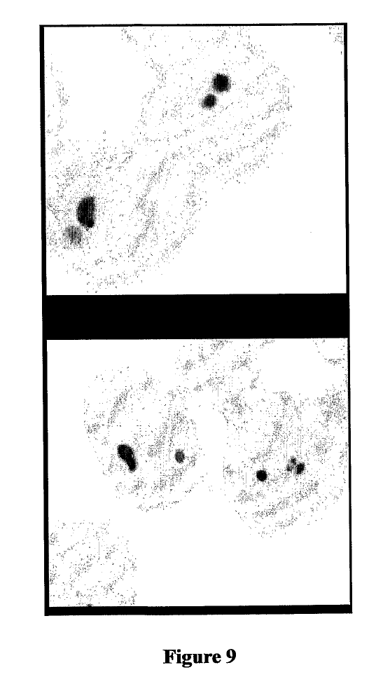

As can be seen from FIG. 9, male fetal cells were double stained for X and Y-chromosome markers showing that anti-telomerase antibodies can be used to isolate fetal cells from maternal blood.

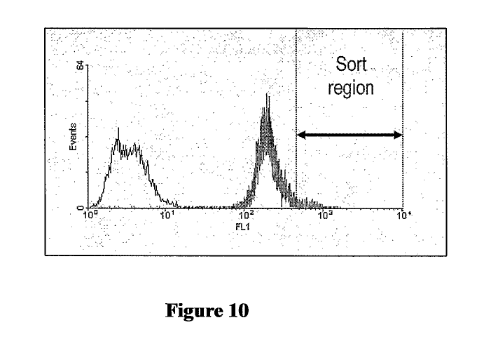

In a further experiment, whole blood samples (10 ml) were depleted of erthyrocytes by density gradient centrifugation, labeled with anti-telomerase polyclonal antibody and analysed by FACS. Cells in the region encompassing the top 5% of fluorescence intensities were sorted (FIG. 10) and assessed for fetal cell content by fluorescence in situ hybridization.

A study of 10 samples gave male fetal cells in 50% of cases with fetal cell numbers ranging from 1-10. These figures are similar to those obtained using the HLA negative selection approach described in Example 3.

### Example 8

### Combined Depletion of Maternal Cells Expressing MHC and Selection of Cells Based on Expression of Telomerase and/or Telomere Length

Two protocols are provided below, however, as the skilled person would be aware many of individual procedures described below are interchangeable between the two protocols. Considering the present disclosure, other protocols can readily be devised.

### Protocol 1

Cells from blood of a pregnant female are separated from plasma by centrifugation. Red cells are depleted on Percoll density gradients. Cells are fixed and permeabilized using a commercial kit—DAKO-Intrastain. The cells are washed again in PBS, and then incubated with monoclonal anti-telomerase antibody (Abcam Ltd, Cambridge, UK) for 1 hour at room temperature.

The cells are then washed in PBS (150 mM NaCl, 10 mM phosphate buffer) containing 0.5% bovine serum albumin (BSA), and a Fluorescein Isothiocyanate (FITC) fluorescently labelled secondary antibody which binds the monoclonal antibody is added for 1 hour at room temperature. Cells are washed in PBS containing 0.5% BSA.

Cells are analysed and labelled cells separated using fluorescence activated cell sorting on a MoFlo High-speed cell sorter (Dako-Cytomation, Ltd).

Following the above positive selection, the enriched fetal cell population is depleted for at least some of the remaining maternal cells expressing MHC molecules. To achieve this end, cells are exposed to saturating amounts of the following biotinylated antibodies against a HLA Class 1 epitope common to all HLA-A, B and C:
a. US Biological; Cat # H6098-39F2; Mouse anti-Human HLA Class 1 Antigen ABC; (Data code: F2)
b. USBiological; Cat# H6098-60B; Mouse anti-Human HLA Class 1 Antigen ABC (Data code: 60B), and
c. EBioscience; Cat# 13-9983-82; Mouse anti-Human HLA Class 1 Antigen ABC; clone W6/32 (Data code: W6).

Cells are then washed and labelled with saturating amounts of streptavidin-coated paramagnetic particles (Molecular Probes/Invitrogen; Cat# C-21476 “Captivate”). Magnetically labelled cell samples are passed through a magnetised column (Miltenyi, LS columns Cat# 130-042-401), retaining all labelled cells. Cells passing through the column include the further enriched fetal cell population and are collected for further analysis.

Analysis to confirm the presence of fetal cells may be by Fluorescence in situ hybridisation or by quantitative PCR

### Protocol 2

Maternal blood samples (8-16 ml) are drawn into vacuum collection tubes with EDTA as anti-coagulant. The samples are processed either fresh or after overnight storage at 4° C.

Mononuclear cells are isolated by density gradient (Ficoll 1.083) centrifugation, and the entire samples are magnetically labelled with antibodies against epitopes on HLA-B locus: one Lambda; mouse anti-human Bw4 (cat #BIH0007; mouse anti-human IgG2a); mouse anti-human Bw6 (cat #BIH0038; mouse anti-human IgG3) (Data code Bw4/6).

Cells are then washed and labelled with saturating amounts of streptavidin-coated paramagnetic particles (Molecular Probes/Invitrogen; Cat# C-21476 “Captivate”). Magnetically labelled cell samples are passed through a magnetised column (Miltenyi, LS columns Cat# 130-042-401), retaining all labelled cells. Cells passing through the column are collected for further processing for telomere length.

Following the above negative selection, fetal cells in the enriched fetal cell population are selected on the basis of telomere length. To achieve this end, cells are washed once in 500 μl TE and centrifuged at 1000 g for 5 min. Cells are resuspended in 5 μl of TE (avoiding bubbles). 20 μl of PNA (Dako Telomere PNA kit/FITC, Dako-Cytomation) in hybridization buffer is added and co-denatured at 80° C. for 20 min in thermocycler.

Hybridization is allowed to proceed for 12 hours at 37° C. The cells are then washed with 2×SSC buffer, and pelleted at 1000 g for 5 min. As much supernatant as possible is removed, and the cells resuspended in 200 μl 2×SSC/0.3% NP40. The cells are incubated at 37° C. for 30 min, centrifuged at 1000 g for 5 min, and as much supernatant as possible removed. The cells are then resuspended in 200 μl 2×SSC/0.3% NP40, and incubated at room temp for 30 min. The cells are then centrifuged at 1000 g for 5 min. As much supernatant as possible is removed and the cells resuspended in 2.5 μl TE.

Cells are analysed and labelled cells separated using fluorescence activated cell sorting on a MoFlo High-speed cell sorter (Dako-Cytomation, Ltd).

Analysis to confirm the presence of fetal cells may be by Fluorescence in situ hybridisation or by quantitative PCR

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Al-Mufti, R. et al. (1999) Am. J. Med. Genet. 85:66-75.
Baerlocher et al. (2002) Cytometry 47: 89-99.
Baerlocher et al. (2003) Cytometry 55A: 1-6.
Bauer et al. (2002) Int. J. Legal Med. 116:39-42.
Berglund and Starkey (1989) J Immunol Methods 125: 79-87.
Blaschitz et al. (2000) Placenta 21:733-741.
Cabuy et al. (2004) Cytometry 62A:150-161.
De Pauw et al. (1998) Cytometry 32:163-169.
Douglas et al. (1959) Am. J. Obstet. Gynec. 78:960-973.
Findlay et al. (1996) Hum Reprod Update 2: 137-152.
Findlay et al. (1998) J Clin Pathol Mol Pathol 51: 164-167.
Findlay et al. (2001) Mol Cellul Endocrinol 183: S5-S12.
Fitzgerald et al. (1993) Biotechniques 15:128-133.
Harrington et al. (1997) Science 275: 973-977.
Kavaler et al. (1998) Cancer 82: 708-714.
King et al. (1996) The Journal of Immunology 156:2068-2076.
Lehman and Kreipe (2001) Methods 25:409-418.
Matthews et al. (2001) J Path 194: 183-193.
Michalet et al. (2005) Science 307:538-544.
Narath et al. (2005) Cytometry 68A: 113-120.
Satyanarayana et al. (2004) Cell Cycle 3: 1138-1150.
Schmid et al. (2002) Cytometry 49: 96-105.
Schroder (1975) J. Med. Genet. 12:230-242.
Seimiya et al. (2000) EMBO J 19: 2652-2661.
Seki et al. (2001) Clinical Cancer Research 7: 1976-1981.
Shorter et al. (1993) Placenta 14:571-582.
Sidransky (2002) Nature Reviews Cancer 2: 210-219.
Tokumasu and Dvorak (2003) J. Microsc. 211:256-261.
Trulsson et al. (2003) Am J Surg 186: 83-88.
Vaziri et al. (1994) PNAS 91:9857-9860.

1. A method of enriching fetal cells from a sample, the method comprising;
i) depleting maternal cells from a sample by removing cells that express at least one MHC molecule on the cells' surface, and
ii) selecting fetal cells from the sample by at least one of:
a) selecting cells that express telomerase, and
b) selecting cells based on telomere length.

2. A method of enriching fetal cells from a sample, the method comprising removing from the sample cells that express at least one MHC molecule on the cells' surface.

3. The method of claim 1, wherein the MHC molecule is a Class I MHC molecule.

4. The method of claim 3, wherein the Class I MHC molecule is at least one of HLA-A and HLA-B.

5. The method of claim 4, wherein one method for removing cells that express at least one MHC molecule on the cell surface comprises:
i) contacting cells in the sample with an agent that binds at least one MHC molecule and
ii) removing cells bound by the agent.

6-7. (canceled)

8. The method of claim 5, wherein the method comprises contacting the sample with i) an agent that binds at least one Class I MHC molecule, and ii) an agent that binds at least one Class II MHC molecule.

9. The method of claim 5, wherein the agent binds:
i) a monomorphic determinant of HLA-A molecules,
ii) a monomorphic determinant of HLA-B molecules or
iii) a monomorphic determinant of HLA-A and HLA-B molecules.

10-11. (canceled)

12. The method of claim 5, wherein the agent is used at sub-saturating concentrations.

13-24. (canceled)

25. A method of enriching fetal cells from a sample, the method comprising selecting cells from the sample that express telomerase.

26. The method of claim 1, wherein the method comprises at least one of: detecting a protein component of telomerase; detecting an RNA component of telomerase; and detecting an mRNA encoding a protein component of telomerase.

27-34. (canceled)

35. A method of enriching fetal cells from a sample, the method comprising selecting cells based on telomere length.

36-37. (canceled)

38. The method claim 1, wherein the sample is maternal blood, cervical mucous or urine.

39-44. (canceled)

45. A method of detecting a fetal cell(s) in a sample, the method comprising at least one of: analysing a candidate cell for the expression of telomerase; analysing a candidate cell for the presence of telomeres; and analysing a candidate cell for the length of the telomeres.

46. (canceled)

47. The method of claim 1, further comprising; obtaining an enriched population of fetal cells.

48. The method of claim 47, further comprising adding a carrier to the enriched population of fetal cells.

49. A method for enriching fetal cells from a sample comprising: using an agent that binds at least one MHC molecule, using an agent that binds a compound that associates with an MHC molecule, or using an agent that binds telomerase or telomeres, or using a combination thereof for enriching the fetal cells from the sample.

50-51. (canceled)

52. The method of claim 1, further comprising: analysing the genotype of a fetal cell at a locus of interest, the method comprising:
i) obtaining enriched fetal cells using at least one of the method of claim 1, and detecting a fetal cell using a method comprising at least one of: analysing a candidate cell for the expression of telomerase, analysing a candidate cell for the presence of telomeres, and analysing a candidate cell for the length of the telomeres; and
ii) analysing the genotype of at least one fetal cell at a locus of interest.

53-54. (canceled)

55. The method of claim 1 further comprising: determining the sex of a fetus, the method comprising:
i) obtaining enriched fetal cells using at least one of the method of claim 1; and detecting a fetal cell using a method comprising at least one of: analysing a candidate cell for the expression of telomerase, analysing a candidate cell for the presence of telomeres, and analysing a candidate cell for the length of the telomeres; and
ii) analysing at least one fetal cell to determine the sex of the fetus.

56. The method of claim 1 further comprising: determining the father of a fetus, the method comprising:
i) obtaining enriched fetal cells using at least one of the method of claim 1, and detecting a fetal cell using a method comprising at least one of: analysing a candidate cell for the expression of telomerase, analysing a candidate cell for the presence of telomeres, and analysing a candidate cell for the length of the telomeres; and
ii) determining the genotype of the candidate father at one or more loci,
iii) determining the genotype of the fetus at one or more of the loci, and
iv) comparing the genotypes of ii) and iii) to determine the probability that the candidate father is the biological father of the fetus.

57. (canceled)

58. A kit for enriching fetal cells from a sample, the kit comprising:
i) at least one of an agent that binds at least one MHC molecule, an agent that binds a compound that associates with an MHC molecule; and an agent that binds a hemopoietic cell; and
ii) at least one of a molecule which binds to telomerase; a molecule which hybridizes to a polynucleotide encoding a protein component of the telomerase, and a molecule which hybridizes to telomeres.

59. A kit for enriching fetal cells from a sample, the kit comprising at least one of an agent that binds at least one MHC molecule; an agent that binds a compound that associates with an MHC molecule; and an agent that binds a hemopoietic cell.

60-62. (canceled)

63. A kit for detecting a fetal cell, the kit comprising at least one of a molecule which binds to telomerase; a molecule which hybridizes to a polynucleotide encoding a protein component of the telomerase; and a molecule which hybridizes to telomeres.

64. (canceled)

65. A kit for detecting a genetic abnormality in a fetal cell, the kit comprising:
i) a molecule for detecting a fetal cell, wherein the molecule binds to telomerase, which hybridizes to a polynucleotide encoding a protein component of the telomerase, or which hybridizes to telomeres; and
ii) at least one reagent for detecting the genetic abnormality.


SEQUENCE LISTING (3 sequences)

SEQ ID NO: 1 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 2 - RNA, 4015 nt, Homo sapiens
gcagcgcugcguccugcugcgcacgugggaagcccuggccccggccacccccgcgaugccgcgcgcuccccgcugccgagccgugcgcucccugcugcgcagccacuaccgcgaggugcugccgcuggccacguucgugcggcgccuggggccccagggcuggcggcuggugcagcgcggggacccggcggcuuuccgcgcgcugguggcccagugccuggugugcgugcccugggacgcacggccgccccccgccgcccccuccuuccgccagguguccugccugaaggagcugguggcccgagugcugcagaggcugugcgagcgcggcgcgaagaacgugcuggccuucggcuucgcgcugcuggacggggcccgcgggggcccccccgaggccuucaccaccagcgugcgcagcuaccugcccaacacggugaccgacgcacugcgggggagcggggcgugggggcugcugcugcgccgcgugggcgacgacgugcugguucaccugcuggcacgcugcgcgcucuuugugcugguggcucccagcugcgccuaccaggugugcgggccgccgcuguaccagcucggcgcugccacucaggcccggcccccgccacacgcuaguggaccccgaaggcgucugggaugcgaacgggccuggaaccauagcgucagggaggccggggucccccugggccugccagccccgggugcgaggaggcgcgggggcagugccagccgaagucugccguugcccaagaggcccaggcguggcgcugccccugagccggagcggacgcccguugggcagggguccugggcccacccgggcaggacgcguggaccgagugaccgugguuucuguguggugucaccugccagacccgccgaagaagccaccucuuuggagggugcgcucucuggcacgcgccacucccacccauccgugggccgccagcaccacgcgggccccccauccacaucgcggccaccacgucccugggacacgccuugucccccgguguacgccgagaccaagcacuuccucuacuccucaggcgacaaggagcagcugcggcccuccuuccuacucagcucucugaggcccagccugacuggcgcucggaggcucguggagaccaucuuucuggguuccaggcccuggaugccagggacuccccgcagguugccccgccugccccagcgcuacuggcaaaugcggccccuguuucuggagcugcuugggaaccacgcgcagugccccuacggggugcuccucaagacgcacugcccgcugcgagcugcggucaccccagcagccggugucugugcccgggagaagccccagggcucuguggcggcccccgaggaggaggacacagacccccgucgccuggugcagcugcuccgccagcacagcagccccuggcagguguacggcuucgugcgggccugccugcgccggcuggugcccccaggccucuggggcuccaggcacaacgaacgccgcuuccucaggaacaccaagaaguucaucucccuggggaagcaugccaagcucucgcugcaggagcugacguggaagaugagcgugcgggacugcgcuuggcugcgcaggagcccagggguuggcuguguuccggccgcagagcaccgucugcgugaggagauccuggccaaguuccugcacuggcugaugaguguguacgucgucgagcugcucaggucuuucuuuuaugucacggagaccacguuucaaaagaacaggcucuuuuucuaccggaagagugucuggagcaaguugcaaagcauuggaaucagacagcacuugaagagggugcagcugcgggagcugucggaagcagaggucaggcagcaucgggaagccaggcccgcccugcugacguccagacuccgcuucauccccaagccugacgggcugcggccgauugugaacauggacuacgucgugggagccagaacguuccgcagagaaaagagggccgagcgucucaccucgagggugaaggcacuguucagcgugcucaacuacgagcgggcgcggcgccccggccuccugggcgccucugugcugggccuggacgauauccacagggccuggcgcaccuucgugcugcgugugcgggcccaggacccgccgccugagcuguacuuugucaagguggaugugacgggcgcguacgacaccaucccccaggacaggcucacggaggucaucgccagcaucaucaaaccccagaacacguacugcgugcgucgguaugccgugguccagaaggccgcccaugggcacguccgcaaggccuucaagagccacgucucuaccuugacagaccuccagccguacaugcgacaguucguggcucaccugcaggagaccagcccgcugagggaugccgucgucaucgagcagagcuccucccugaaugaggccagcaguggccucuucgacgucuuccuacgcuucaugugccaccacgccgugcgcaucaggggcaaguccuacguccagugccaggggaucccgcagggcuccauccucuccacgcugcucugcagccugugcuacggcgacauggagaacaagcuguuugcggggauucggcgggacgggcugcuccugcguuugguggaugauuucuuguuggugacaccucaccucacccacgcgaaaaccuuccucaggacccugguccgaggugucccugaguauggcugcguggugaacuugcggaagacaguggugaacuucccuguagaagacgaggcccuggguggcacggcuuuuguucagaugccggcccacggccuauuccccuggugcggccugcugcuggauacccggacccuggaggugcagagcgacuacuccagcuaugcccggaccuccaucagagccagucucaccuucaaccgcggcuucaaggcugggaggaacaugcgucgcaaacucuuuggggucuugcggcugaagugucacagccuguuucuggauuugcaggugaacagccuccagacggugugcaccaacaucuacaagauccuccugcugcaggcguacagguuucacgcaugugugcugcagcucccauuucaucagcaaguuuggaagaaccccacauuuuuccugcgcgucaucucugacacggccucccucugcuacuccauccugaaagccaagaacgcagggaugucgcugggggccaagggcgccgccggcccucugcccuccgaggccgugcaguggcugugccaccaagcauuccugcucaagcugacucgacaccgugucaccuacgugccacuccuggggucacucaggacagcccagacgcagcugagucggaagcucccggggacgacgcugacugcccuggaggccgcagccaacccggcacugcccucagacuucaagaccauccuggacugauggccacccgcccacagccaggccgagagcagacaccagcagcccugucacgccgggcucuacgucccagggagggaggggcggcccacacccaggcccgcaccgcugggagucugaggccugagugaguguuuggccgaggccugcauguccggcugaaggcugaguguccggcugaggccugagcgaguguccagccaagggcugaguguccagcacaccugccgucuucacuuccccacaggcuggcgcucggcuccaccccagggccagcuuuuccucaccaggagcccggcuuccacuccccacauaggaauaguccauccccagauucgccauuguucaccccucgcccugcccuccuuugccuuccacccccaccauccagguggagacccugagaaggacccugggagcucugggaauuuggagugaccaaaggugugcccuguacacaggcgaggacccugcaccuggaugggggucccugugggucaaauuggggggaggugcugugggaguaaaauacugaauauaugaguuuuucaguuuugaaaaaaa

SEQ ID NO: 3 - RNA, 451 nt, Homo sapiens
ggguugcggagggugggccugggaggggugguggccauuuuuugucuaacccuaacugagaagggcguaggcgccgugcuuuugcuccccgcgcgcuguuuuucucgcugacuuucagcgggcggaaaagccucggccugccgccuuccaccguucauucuagagcaaacaaaaaaugucagcugcuggcccguucgccccucccggggaccugcggcgggucgccugcccagcccccgaaccccgccuggaggccgcggucggcccggggcuucuccggaggcacccacugccaccgcgaagaguugggcucugucagccgcgggucucucgggggcgagggcgagguucaggccuuucaggccgcaggaagaggaacggagcgaguccccgcgcgcggcgcgauucccugagcugugggacgugcacccaggacucggcucacacaugc